(19) United States
(12) Patent Application Publication
(10) Pub. No.: US 2016/0175485 A1
(43) Pub. Date: Jun. 23, 2016

(54) COMBINATION THERAPY TO PROMOTE WOUND HEALING

(71) Applicant: THE REGENTS OF THE UNIVERSITY OF CALIFORNIA, Oakland, CA (US)

(72) Inventors: Roslyn Rivkah ISSEROFF, Davis, CA (US); Ravi Krishna Mohan DASU, Davis, CA (US); Sandra RAMIREZ, Davis, CA (US)

(73) Assignee: The Regents of the University of California, Oakland, CA (US)

(21) Appl. No.: 14/913,284

(22) PCT Filed: Aug. 19, 2014
(86) PCT No.: PCT/US2014/051723

§ 371 Date: Feb. 19, 2016

Related U.S. Application Data
(60) Provisional application No. 61/870,766, filed on Aug. 27, 2013.

(51) Int. Cl. A61L 27/36; A61L 27/54; A61L 27/60; A61K 35/28; A61L 27/38

(52) U.S. Cl. CPC A61L 27/3633; A61K 35/28; A61L 27/3834; A61L 27/3895; A61L 27/60; A61L 27/54; A61L 2300/436; A61L 2430/34

(57) ABSTRACT

Methods for increasing and/or promoting wound healing, wound re-epithelialization and dermal regeneration of epithelial tissues and cutaneous wounds by administration to a subject of an extracellular matrix scaffold or Scaffold for Dermal Regeneration (SDR) populated with beta adrenergic receptor antagonist pre-conditioned mesenchymal stem cells (MSCs) are provided. Compositions and kits comprising an extracellular matrix scaffold (ECMS) populated with beta adrenergic receptor antagonist pre-conditioned mesenchymal stem cells (MSCs) are also provided.

### CROSS-REFERENCE TO RELATED APPLICATIONS

This application is a U.S. national phase filing under 35 U.S.C. §371 of Intl. Appl. No. PCT/US2014/051723, filed on Aug. 9, 2014, which claims the benefit under 35 U.S.C. §119(e) of U.S. Provisional Application No. 61/870,766, filed on Aug. 27, 2013, each of which are hereby incorporated herein by reference in their entireties for all purposes.

### FIELD

Methods for increasing and/or promoting wound healing, wound re-epithelialization and dermal regeneration of epithelial tissues and cutaneous wounds by administration to a subject of an extracellular matrix scaffold or Scaffold for Dermal Regeneration (SDR) populated with beta adrenergic receptor antagonist pre-conditioned mesenchymal stem cells (MSCs) are provided. Compositions and kits comprising an extracellular matrix scaffold or Scaffold for Dermal Regeneration (SDR) populated with beta adrenergic receptor antagonist pre-conditioned mesenchymal stem cells (MSCs) are also provided.

### BACKGROUND

Stress plays an important role in wound healing and some of the most potent mediators of stress are the catecholamines, such as epinephrine and norepinephrine. It has been shown that epinephrine can activate beta adrenergic receptors on human keratinocytes causing them to release inflammatory mediators such as IL-6. An estimated 40-60% of diabetes (DB) patients are at risk for the development of DB foot complications, and DB foot wounds (DFW) account for over 20% of all hospitalizations of DB patients. These wounds are extremely unmanageable resulting in an estimated 82,000 non-traumatic lower limb amputations each year, in other words, one amputation every 30 seconds in DB patients.

Current topical methods for treating DFW includes debridement to remove necrotic and infected tissues, dressings to provide a moist wound environment, bandages, and topical applications of antimicrobial or biologic agents, offloading, physical therapies, and educational strategies. However, these different treatment modalities often fail to achieve complete wound closure since they do not address the main culprit, i.e., persistent inflammation. For example excessive use of antibiotics may address bacterial numbers and to some extent inflammation but can lead to the development of resistant strains.

### SUMMARY

In one aspect, provided are extracellular matrix scaffolds. In some embodiments, the extracellular matrix scaffolds comprise mesenchymal stem cells (MSCs) which have been contacted and/or pre-conditioned with and/or exposed to a beta adrenergic receptor antagonist. In some embodiments, the MSCs have been cultured in medium comprising a beta adrenergic receptor antagonist. In varying embodiments, the MSCs have been cultured in medium comprising a beta adrenergic receptor antagonist at a concentration in the range of about 0.2 μM to about 50 μM, e.g., about 0.4 μM to about 40 μM, e.g., about 0.3 μM to about 30 μM, e.g., about 0.2 μM to about 20 μM, e.g., about 1.0 μM to about 10 μM. In varying embodiments, the MSCs have been cultured at least 24 hours, e.g., at least about 48 hours in medium comprising a beta adrenergic receptor antagonist. In varying embodiments, the MSCs have been cultured under hypoxic conditions. In some embodiments, the antagonist has a Kd for a beta-3 adrenergic receptor that is about 100 or more times greater than a Kd of the antagonist for a non-beta-3 (e.g., for a β1 and/or β2) adrenergic receptor. In some embodiments, the beta adrenergic receptor antagonist is non-selective antagonist for β1 and β2 adrenergic receptors. In some embodiments, the beta adrenergic receptor antagonist is selected from carteolol, carvedilol, labetalol, nadolol, penbutolol, pindolol, propranolol, sotalol, timolol, and mixtures, analogs and salts thereof. In some embodiments, the beta adrenergic receptor antagonist is selective antagonist for β1 adrenergic receptors. In some embodiments, the beta adrenergic receptor antagonist is selected from acebutolol, atenolol, betaxolol, bisoprolol, celiprolol, esmolol, metoprolol, nebivolol, and mixtures, analogs and salts thereof. In some embodiments, the beta adrenergic receptor antagonist is selective antagonist for β2 adrenergic receptor. In some embodiments, the selective antagonist for β2 adrenergic receptor is selected from butoxamine and ICI-118,551. In some embodiments, the beta adrenergic receptor antagonist is selected from the group consisting of timolol, labetalol, dilevelol, propanolol, carvedilol, nadolol, carteolol, penbutolol, sotalol, ICI-118,551, butoxamine, and mixtures, analogs and salts thereof. In some embodiments, the beta adrenergic receptor antagonist is substantially free of activity as a beta-3 adrenergic receptor agonist. In some embodiments, the beta adrenergic receptor antagonist is attached to, e.g., via covalent bonding or crosslinking, to the scaffold. In varying embodiments, the MSCs are adipose-derived MSCs (Ad-MSCs). In varying embodiments, the MSCs are bone-marrow-derived MSCs (BM-MSCs).

In a further aspect, provided are kits comprising an extracellular matrix scaffold as described above and herein.

In another aspect, provided are methods of promoting, facilitating, and/or increasing healing, closure, re-epithelization and/or dermal regeneration of an epithelial and/or cutaneous wound in a subject in need thereof, comprising placing, implanting, suturing or embedding onto or into the wound an extracellular matrix scaffold as described above and herein. In varying embodiments, the subject has diabetes. In some embodiments, the subject is a human. In some embodiments, the wound comprises an incision, a laceration, an abrasion, or an ulcer. In some embodiments, the wound is a chronic wound. In some embodiments, the wound comprises a venous stasis ulcer, a diabetic foot ulcer, a neuropathic ulcer, or a decubitus ulcer. In some embodiments, the wound comprises a wound resulting from surgical wound dehiscence. In some embodiments, the wound comprises a burn. In some embodiments, the epithelial wound comprises skin. In varying embodiments, the MSCs are syngeneic to the subject. In varying embodiments, the MSCs are autologous to the subject. In varying embodiments, the MSCs are allogeneic to the subject. In varying embodiments, the MSCs are xenogeneic to the subject. In varying embodiments, the wound is sterile. In varying embodiments, the wound is not sterile. In varying embodiments, the beta adrenergic receptor antagonist is applied multiple times to the extracellular matrix scaffold that has been sutured, embedded or implanted into the wound.

### DEFINITIONS

Unless defined otherwise, all technical and scientific terms used herein have the same meaning as commonly understood by one of ordinary skill in the art. The following definitions supplement those in the art and are directed to the current application and are not to be imputed to any related or unrelated case, e.g., to any commonly owned patent or application. Although any methods and materials similar or equivalent to those described herein can be used in the practice for testing, the preferred materials and methods are described herein. Accordingly, the terminology used herein is for the purpose of describing particular embodiments only, and is not intended to be limiting.

As used in this specification and the appended claims, the singular forms “a,” “an” and “the” include plural referents unless the context clearly dictates otherwise. Thus, for example, reference to “a receptor” includes a plurality of receptors; reference to “a cell” includes mixtures of cells, and the like.

The term “about” as used herein indicates the value of a given quantity varies by +/−10% of the value, or optionally +/−5% of the value, or in some embodiments, by +/−1% of the value so described.

The term “topical” refers to administration or delivery of a compound (e.g., a beta adrenergic receptor antagonist) by application of the compound to a surface of a body part. For example, a compound can be topically administered by applying it to skin, to the surface of a wound within the skin, a mucus membrane, or wound within the mucous membrane, or another body surface, or wound within. Topical administration can result, e.g., in either local or systemic delivery of a compound.

An “antagonist” is a compound (e.g., a drug) that can bind to a receptor and prevent an agonist from binding to and activating that receptor. Typically, binding of an antagonist to a receptor forms a complex which does not give rise to any response, as if the receptor were unoccupied. Alternatively, the antagonist can be a partial agonist.

It is worth noting that certain compounds can be classified as both an agonist and an antagonist. For example, a “mixed agonist-antagonist” (also called a “partial agonist”) is a compound which possesses affinity for a receptor, but which, unlike a full agonist, will elicit only a small degree of the response characteristic of that receptor, even if a high proportion of receptors are occupied by the compound. Such occupancy of the receptors by the partial agonist can prevent binding of a full agonist (e.g., an endogenous agonist) to the receptor.

The term “co-administering” or “concurrent administration”, when used, for example with respect to the compounds (e.g., one or more antagonists of a beta-adrenergic receptor) and/or analogs thereof and another active agent (e.g., an anesthetic, an antibiotic), refers to administration of the compound and/or analogs and the active agent such that both are in the blood at the same time. Co-administration can be concurrent or sequential.

The term “effective amount” or “pharmaceutically effective amount” refer to the amount and/or dosage, and/or dosage regimen of one or more compounds necessary to bring about the desired result e.g., an amount sufficient to promote, increase and/or facilitate wound healing, closure, re-epithelialization and/or dermal regeneration of an epithelial or cutaneous wound in a subject.

The phrase “cause to be administered” refers to the actions taken by a medical professional (e.g., a physician), or a person controlling medical care of a subject, that control and/or permit the administration of the agent(s)/compound(s) at issue to the subject. Causing to be administered can involve diagnosis and/or determination of an appropriate therapeutic or prophylactic regimen, and/or prescribing particular agent(s)/compounds for a subject. Such prescribing can include, for example, drafting a prescription form, annotating a medical record, and the like.

As used herein, the terms “treating” and “treatment” refer to delaying the onset of, retarding or reversing the progress of, reducing the severity of, or alleviating or preventing either the disease or condition to which the term applies (e.g., epithelial and/or cutaneous wound healing, closure, re-epithelialization and/or dermal regeneration), or one or more symptoms of such disease or condition.

As used herein, the phrase “consisting essentially of” refers to the genera or species of active pharmaceutical agents recited in a method or composition, and further can include other agents that, on their own do not have substantial activity for the recited indication or purpose.

The terms “subject,” “individual,” and “patient” interchangeably refer to any mammal, including humans and non-human mammals, e.g., primates, domesticated mammals (e.g., canines and felines), agricultural mammals (e.g., bovines, ovines, equines, porcines) and laboratory mammals (e.g., rats, mice, rabbits, guinea pigs, hamsters), as described herein.

The terms “increasing,” “promoting,” “enhancing” with respect to wound healing refers to increasing the epithelialization, closure and/or dermal regeneration of a wound in a subject by a measurable amount using any method known in the art. The wound healing is increased, promoted or enhanced if the re-epithelialization, closure and/or dermal regeneration of the wound is at least about 10%, 20%, 30%, 50%, 80%, or 100% increased in comparison to the re-epithelialization, closure and/or dermal regeneration of the wound prior to administration of beta adrenergic receptor antagonist conditioned mesenchymal stem cells (MSCs), e.g., over a predetermined time period. In some embodiments, the re-epithelialization, closure and/or dermal regeneration of the wound is increased, promoted or enhanced by at least about 1-fold, 2-fold, 3-fold, 4-fold, or more in comparison to the re-epithelialization, closure and/or dermal regeneration of the wound prior to administration of the beta adrenergic receptor antagonist conditioned MSCs.

The terms “reducing,” “decreasing” with respect to wound size refers to reducing or decreasing the open wound surface area or the wound volume in a subject by a measurable amount using any method known in the art. The wound surface area or volume in a subject is reduced or decreased if the measurable parameter of the wound is at least about 10%, 20%, 30%, 50%, 80%, or 100% reduced or decreased in comparison to the measurable parameter of the one or more symptoms prior to administration of the beta adrenergic receptor antagonist conditioned MSCs. In some embodiments, the measurable parameter of the wound surface area or volume is reduced or decreased by at least about 1-fold, 2-fold, 3-fold, 4-fold, or more in comparison to the measurable parameter of the one or more symptoms prior to administration of the beta adrenergic receptor antagonist conditioned MSCs.

The term “mesenchymal stem cells” refers to stem cells defined by their capacity to differentiate into bone, cartilage, and adipose tissue. With respect to cell surface markers, MSCs generally express CD44 and CD90, and should not express CD34, CD45, CD80, CD86 or MHC-II.

### BRIEF DESCRIPTION OF THE DRAWINGS

FIGS. 1A-B illustrate that hypoxia conditioned bone marrow mesenchymal stem cells (BM-MSCs) improve wound healing in healing-impaired diabetic mice. Full thickness cutaneous excisional wounds were created on the backs of db/db mice as previously described. Immediately, scaffolds for dermal regeneration (SDR, in this case, Integra™) under three different conditions were implanted onto the wounds. The three conditions are (1) SDR with MSC, (2) SDR with MSC and, (3) MSC-containing SDR cultured for 2 days under hypoxic conditions. Wound re-epithelialization was quantified at day 9 in H&E stained wound sections. (A) Representative H&E stained images; (B) Average percent wound re-epithelialization (mean±SD). *P<0.02 SDR+MSC vs. SDR+MSC, +hypoxia, n=5-7 mice/group.

FIGS. 2A-B illustrate that timolol pre-conditioning of MSC-containing SDR promotes wound closure in healing impaired diabetic mice. Full thickness cutaneous excisional wounds were created on the backs of db/db mice as previously described. Immediately, scaffolds for dermal regeneration (SDR, Integra™) under three different conditions were implanted onto the wounds. The three conditions are (1) SDR with MSC, (2) MSC-containing SDR cultured for 2 days in 1 μM timolol and, (3) MSC-containing SDR cultured for 2 days in 10 μM timolol. Wound re-epithelialization was quantified at day 9 in H&E stained wound sections. (A) Representative H&E stained images; (B) Average percent wound re-epithelialization (mean±SD). *P<0.02 vs. SDR+MSC, n=4 mice/group.

FIGS. 3A-B illustrate that a combination of hypoxia and timolol pre-conditioning of MSC-containing SDR promotes wound closure in healing impaired diabetic mice. Full thickness cutaneous excisional wounds were created on the backs of db/db mice. Immediately, scaffolds for dermal regeneration (SDR, in this case, Integra™) under three different conditions were implanted on to the wounds. The three conditions are (1) SDR with timolol [1 μM] (2) MSC-containing SDR cultured for 2 days in 1 μM timolol and, (3) MSC-containing SDR cultured for 2 days 1 μM timolol+hypoxia. Wound re-epithelialization was quantified at day 9 in H&E stained wound sections (A) Representative H&E stained images (n=2 mice) (B) Average percent wound re-epithelialization (mean±SD); n=4 mice/group.

FIGS. 4a-g: Epinephrine (EPI) induces TLR2, MyD88, and IL-6 expression in BM-MSC. a) BM-MSC (passage 3-5) were exposed to EPI [50-1000 nM] and secreted IL-6 levels in the cell culture supernatants were determined by ELISA. Values are expressed as pg/μg protein (mean±SD). *P<0.05 vs 4 hr Control (C); **P<0.05 vs 24 hr Control (n=4). b) TLR2 mRNA expression was measured in EPI [50 nM] treated BM-MSC using RT-PCR. Values are expressed as fold change vs control. MALP2 [100 ng/ml] was used as a positive control. *P<0.05 vs Control (n=4). c) TLR2 and MyD88 protein levels were measured in EPI [50 nM] treated BM-MSC lysates using Western blot assay. GAPDH was used as the loading control and MALP2 [100 ng/ml] as positive control. d) Densitometric analysis of the Western blots. Protein/GAPDH ratio values are expressed as fold change vs control (mean±SD). *P<0.05 vs Control (n=4). FIG. 4e-g: MALP2 induces β2-AR mRNA and protein expression in BM-MSC. e) β2-AR (ADRB2) mRNA expression was measured in MALP2 [100 ng/ml] treated cells using RT-PCR. Values are expressed as fold change (in mRNA/housekeeping gene ratio) vs control (mean±SD). *P<0.001 vs Control (n=3). EPI [50 nM] was used as a positive control. f) β2-AR protein expression was measured in MALP2 [100 ng/ml] treated cells by Western blot. α-tubulin was used as the loading control and EPI [50 nM] as positive control. g) Densitometric analysis of the Western blots. β2-AR/α-tubulin ratio values are expressed as fold change vs control (mean±SD). *P<0.05 vs Control (n=4).

FIGS. 5a-f: Synergistic effects of EPI and MALP2 on β2-AR protein expression and BARK-1 phosphorylation in BM-MSC. a) Western blot analysis of β2-AR protein expression in EPI [50 nM], MALP2 [100 ng/ml] and EPI+MALP2 treated cells. GAPDH was used as the loading control. b) Western blot analysis of BARK-1 phosphorylation in EPI [50 nM], MALP2 [100 ng/ml] and EPI+MALP2 treated cells. Total BARK-1 was used as the internal control. c) Densitometric analysis of the Western blots. β2-AR/GAPDH ratio values are expressed as fold change vs control (mean±SD). *P<0.01 vs Control (n=4). d) Densitometric analysis of the Western blots. pBARK-1/BARK-1 ratio values are expressed as fold change vs control (mean±SD). *P<0.05 vs Control (n=4).

FIG. 5e & f: EPI and MALP2 effects on BM-MSC and NHK single cell migration (SCM). β2-AR and TLR2 activation reduced BM-MSC and NHK migration. Migratory speeds of (e) BM-MSC and (f) NHK plated on collagen-coated glass-bottomed culture dishes and treated with serum free growth medium (control), EPI [50 nM], MALP2 [100 ng/ml], or EPI+MALP2 were determined. SCM rates of at least 50 cells per treatment were measured. Each panel represents the mean±SD of at least 4 experiments (160-200 cells/treatment) using 4 cell strains isolated from four different donors. *P<0.05 vs BM-MSC Control; **P<0.001 vs NHK Control.

FIGS. 6a-f: Synergistic effects of EPI and MALP2 or EPI and heat killed Staphylococcus aureus (HKSA) on IL-6 secretion in BM-MSC and NHK. a & c) BM-MSC were exposed to EPI [50 nM], MALP2 [100 ng/ml], HKSA [10⁴ cells/ml] and EPI+MALP2 or EPI+HKSA for four hours and secreted IL-6 levels in supernatants were determined by ELISA. Values are expressed as pg/μg protein (mean±SD). *P<0.05 vs Control; **P<0.001 vs EPI or MALP2/HKSA, (n=4 experiments). 3b & d) NHK were exposed to EPI [50 nM], MALP2 [100 ng/ml], HKSA [10⁴ cells/ml] and EPI+MALP2 or EPI+HKSA for four hours and secreted IL-6 levels in supernatants were determined by ELISA. Values are expressed as pg/μg protein (mean±SD). *P<0.05 vs Control; **P<0.001 vs EPI or MALP2/HKSA, (n=4 experiments). 3e) Western blot showing the combined effects of EPI+HKSA on TLR2-MyD88 and β2-AR-BARK1 expression in BM-MSC. Cells were exposed to HKSA [10⁴ cells/ml] and EPI+HKSA and total cell protein was subjected to Western blot assay. α-tubulin was used as the loading control and total BARK-1 was used as the internal control for phospho BARK-1 (n=4 experiments). Densitometric ratios (TLR2/α-tubulin, MyD88/α-tubulin, β2-AR/α-tubulin or pBARK-1/BARK-1) are shown in the adjacent table. *P<0.05 vs HKSA. 3f) EPI and HKSA effects on NHK single cell migration. NHK were plated on collagen-coated glass-bottomed culture dishes and treated with HKSA [10⁴ cells/ml] and EPI+HKSA in serum free growth medium and SCM rates of at least 50 cells per treatment were determined. Each panel represents the mean values and standard deviations of at least 4 experiments (200 cells). *P<0.05 vs HKSA (n=4 experiments).

FIGS. 7a-i: Timolol (a non-selective β2-AR antagonist) reverses the combined effects of EPI+MALP2 or EPI+HKSA on SCM and IL-6 secretion in BM-MSC and NHK. a) BM-MSC were exposed to EPI [50 nM]+MALP2 [100 ng/ml] or pretreated with Timolol [10 μM, 30 minutes) and were further treated for four hours with EPI+MALP2. SCM rates of at least 50 cells per treatment were determined. Values are expressed as average migratory speed (μm/min; mean±SD). *P<0.05 vs EPI or MALP2; **P<0.005 vs EPI+MALP2; (n=3 experiments; total of 150 cells). b & c) NHK were exposed to EPI [50 nM]+MALP2 [100 ng/ml], EPI+HKSA [10⁴ cells/ml] or pretreated with Timolol [10 μM, 30 minutes) and were further treated for four hours with EPI+MALP2 or EPI+HKSA. SCM rates of at least 60 cells per treatment were determined. Values are expressed as average migratory speed (μm/min; mean±SD). *P<0.05 vs EPI or MALP2; **P<0.05 vs EPI+MALP2; **P<0.01 vs EPI+HKSA, (n=2 experiments; 120 cells). d & f) BM-MSC were exposed to EPI [50 nM]+MALP2 [100 ng/ml], EPI [50 nM]+HKSA [10⁴ cells/ml] or pretreated with Timolol [10 μM, 30 minutes) and were further treated for four hours with EPI+MALP2 or EPI+HKSA. Secreted IL-6 in the cell culture supernatant was determined using ELISA. Values are expressed as pg/μg protein (mean±SD). *P<0.05 vs EPI or MALP2; **P<0.001 vs EPI+MALP2; **P<0.05 vs EPI+HKSA (n=4 experiments). e & g) NHK were exposed to EPI [50 nM]+MALP2 [100 ng/ml], EPI [50 nM]+HKSA [10⁴ cells/ml] or pretreated with Timolol [10 μM, 30 minutes) and were further treated for four hours with EPI+MALP2 or EPI+HKSA. Secreted IL-6 levels were determined in the cell culture supernatants. Values are expressed as pg/μg protein (mean±SD). *P<0.05 vs EPI or MALP2; **P<0.005 vs EPI+MALP2; **P<0.01 vs EPI+HKSA (n=4 experiments). FIG. 7: Physical interaction between β2-AR and TLR2 signaling pathways in NHKs. h) Western blot showing enhanced expression of β2-AR and pBARK-1 in NHK cell lysates immunoprecipitated with TLR2 antibody after MALP2 [100 ng/ml] challenge. TLR2 was used as an internal/positive/negative controls (n=4 experiments). i) Western blot showing enhanced expression of TLR2, MyD88, and pIRAK-1 in NHK cell lysates immunoprecipitated with β2-AR antibody after EPI [50 nm] challenge. β2-AR and total IRAK-1 were used as internal controls, (n=4 experiments) in addition to the negative controls.

FIGS. 8a-j: MALP2 and HKSA induce catecholamine secretion and catecholamine producing enzymes in BM-MSC/NHKs. a) BM-MSC and b) NHK were stimulated with 100 ng/ml MALP2 in vitro. Cell culture supernatants were collected and analyzed by HPLC for EPI and norepinephrine. All data are presented as pg/μg cell protein (mean±SD). *P<0.05 vs Control (n=4 experiments). c) BM-MSC and d) NHK were stimulated in vitro with 100 ng/ml of MALP2. Total cell protein was isolated and subjected to Western blot analysis for phenylethanolamine N-methyltransferase (PNMT) or Tyrosine hydroxylase (TH) enzymes. α-tubulin was used as a loading control. Densitometric ratios (PNMT/α-tubulin and TH/α-tubulin) are shown in the adjacent table. *P<0.05 vs Control (C) (n=4 experiments). e) Epinephrine and (f) norepinephrine levels in BM-MSCs stimulated with HKSA [10⁴ cells/ml] in vitro. Cell culture supernatants were collected and analyzed by HPLC for EPI and norepinephrine. All data are presented as pg/μg cell protein (mean±SD). *P<0.05 vs Control g) BM-MSC were stimulated in vitro with 10⁴ cells/ml HKSA in vitro. Total cell protein was isolated and subjected to Western blot analysis for phenylethanolamine N-methyltransferase (PNMT) or Tyrosine hydroxylase (TH) enzymes. α-tubulin was used as a loading control. Densitometric ratios (PNMT/α-tubulin and TH/α-tubulin) are shown in the adjacent table. *P<0.05 vs Control (C) (n=4 experiments). h) Epinephrine and (i) norepinephrine levels in NHKs stimulated with HKSA [10⁴ cells/ml] in vitro. Cell culture supernatants were collected and analyzed by HPLC for EPI and norepinephrine. All data are presented as pg/μg cell protein (mean±SD). *P<0.05 vs control (n=3) j) NHK were stimulated in vitro with 10⁴ cells/ml HKSA in vitro. Total cell protein was isolated and subjected to Western blot analysis for phenylethanolamine N-methyltransferase (PNMT) or Tyrosine hydroxylase (TH) enzymes. α-tubulin was used as a loading control. Densitometric ratios (PNMT/α-tubulin and TH/α-tubulin) are shown in the adjacent table. *P<0.05 vs Control (C) (n=4 experiments).

FIGS. 9a-g: Blocking β2-AR with ICI 118,551 or Timolol reverses EPI+MALP2 or EPI+HKSA delayed NHK migration and increased IL-6 production in injured NHK. a) NHK monolayers were pretreated with Timolol or ICI 118,551 (10 μM, 30 minutes) followed by EPI [50 nM], MALP2 [100 ng/ml] or EPI+MALP2 treatment, and then wounded by scratches. The defined areas were photographed at 0 and 12 hours after wounding. The percent wound area closed was calculated and presented in adjacent table along with % change in wound closure (↓=decreased wound closure; ↑=increased wound closure). Values represent mean±SD. *P<0.05 vs Control; **P<0.05 vs EPI+MALP2; §P<0.01 vs MAPL2, (n=3 experiments). b) NHK monolayers were pretreated with Timolol or ICI 118,551 (10 μM, 30 minutes) followed by EPI [50 nM], HKSA [10⁴ cells/ml] or EPI+HKSA treatment, and then wounded by scratches. The defined areas were photographed at 0 and 16 hours after wounding. The percent open and closed wound areas were calculated and presented in bar graph panel. The percent wound area closed was calculated and presented in adjacent table along with % change in wound closure (↓=decreased wound closure; ↑=increased wound closure). Values represent mean±SD. *P<0.05 vs Control; **P<0.05 vs EPI+MALP2; §P<0.01 vs MAPL2, (n=3 experiments). c) NHK monolayers were pretreated with ICI 118,551 or Timolol (10 μM, 30 minutes) followed by EPI [50 nM], MALP2 [100 ng/ml] or EPI+MALP2 treatment, and then wounded by scratches. Cell supernatants were collected for IL-6 ELISA assays. Values represent mean±SD. *P<0.05 vs Control, **P<0.05 vs MALP2, §P<0.05 vs E+MALP2 (n=3 experiments). d) NHK monolayers were pretreated with ICI 118,551 or Timolol (10 μM, 30 minutes) followed by EPI [50 nM], HKSA [10⁴ cells/ml] or EPI+HKSA treatment, and then wounded by scratches. Cell supernatants were collected for IL-6 ELISA analyses. Values represent mean±SD. *P<0.05 vs Control, **P<0.05 vs HKSA, §P<0.05 vs E+HKSA (n=3 experiments). FIG. 9: Full thickness cutaneous wounds of EPI stressed C57BL6J mice show decreased wound closure (e), increased TLR2 protein expression, and decreased ERK1/2 phosphorylation (f), and increased local IL-6 secretion (g). Blocking β2-AR with ICI 118,551 improves healing, decreases IL-6, TLR2 expression, and increases ERK1/2 phosphorylation in vivo. Densitometric ratios (TLR2/α-tubulin and pERK1/2/ERK1/2) are presented below the blots (f). Values represent mean±SD. *P<0.05 vs Control, **P<0.05 vs EPI; §P<0.05 vs EPI; (n=10-15 mice/group).

FIG. 10: Schematic illustrating the cross talk between β2-AR and TLR2 in BM-MSC and NHKs Inflammatory effects of EPI and TLR2 ligands are mediated through phosphorylation of BARK-1 and engagement of MyD88 respectively, leading to decreased cell migration and increased IL-6 secretion. In addition, TLR2 ligands also induce catecholamine secretion by increasing TH and PNMT levels in BM-MSC and NHKs paving way for an autocrine inflammatory loop. Blocking β2-AR with selective (ICI 118,551) or non-selective (Timolol) antagonists can reverse some effects.

FIG. 11 illustrates that results are not limited to one class of beta adrenergic antagonist. Bone marrow MSCs from two different individuals were cultured and treated with either a non-specific beta adrenergic receptor antagonist (timolol) or a beta 2 adrenergic receptor specific antagonist (ICI 118,551) at stated concentrations for 1 hour, then cell migration was tracked for 12 hours. For each experiment 80-90 cells were tracked. Means+/−SEM shown, *=p<0.005. Results demonstrate that both ICI 118,551 and timolol increase migratory speed of bone marrow derived MSC.

FIG. 12 illustrates that exposing MSCs to beta adrenergic receptor antagonist improves healing in pig skin wounds. Provided is an illustrative schema of experimental design. (1) Cell Culture. Human bone marrow MSCs seeded on matrix is cultured in hypoxic (<1% O₂) conditions and 1 μM timolol. (2) Wounding Surgery. Excise 10 mm diameter, 4-5 mm deep circular punch wounds. Implant matrices and topically administer 1 μM Timolol in PBS. Wound is covered with gauze, Tegaderm® and Expandex® shirt. Animal recovers about 9 days post-operation. (3) Tissue Processing. Excise full thickness wounds and fix in 4% paraformaldehyde for 8 days. Sucrose sink in 30% sucrose 5 days. Bisect wound and section to 10 μm thickness. (4) Keratin5 Immunostain & Imaging. Stain dried sections with rabbit pAb anti-Keratin5 (1:500 overnight at 4° C.) and Cy3 conjugated goat anti rabbit (1:500, 1 hr). Serially image and stitch 10× images. (5) Re-epithelialization Analysis. Trace and sum distance of neo-epithelium from wound edge (indicated by marked increased in cell density in dermis). Divide by distance of wound surface from wound edge to another wound edge.

FIG. 13 illustrates pig skin wound re-epithelialization. The results demonstrate that the combination of Matrix+MSC+Hypoxia+timolol improves healing, as compared to matrix alone. Keratin5 staining is in yellow.

FIG. 14 illustrates pig skin wound re-epithelialization. The results demonstrate that the combination of Matrix+MSC+Hypoxia+timolol improves healing, as compared to matrix alone. N=5 in each group. Mean values+/−SEM. P<0.05.

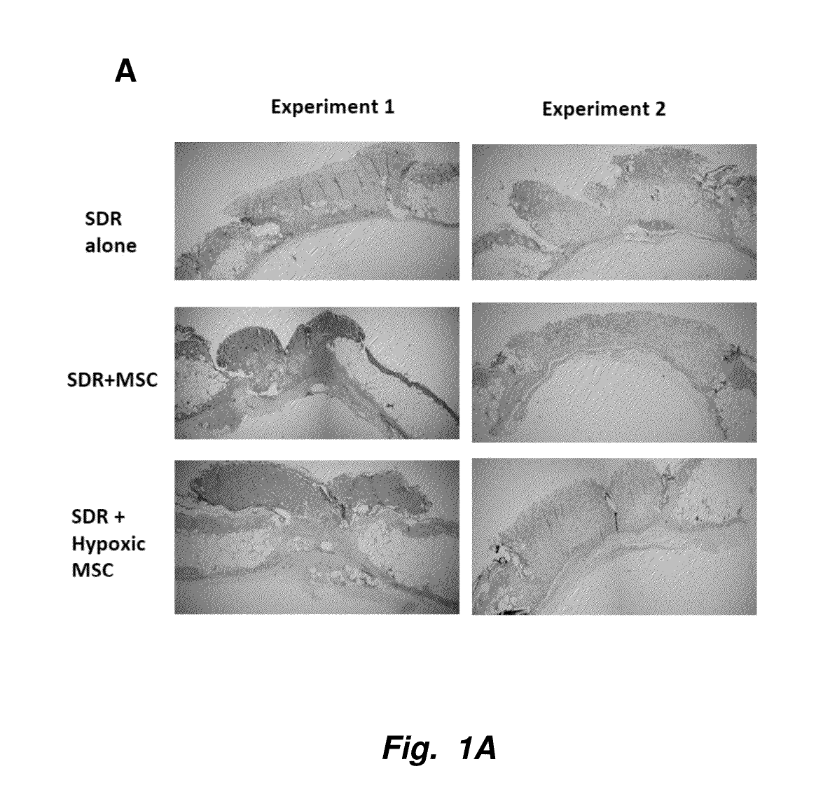

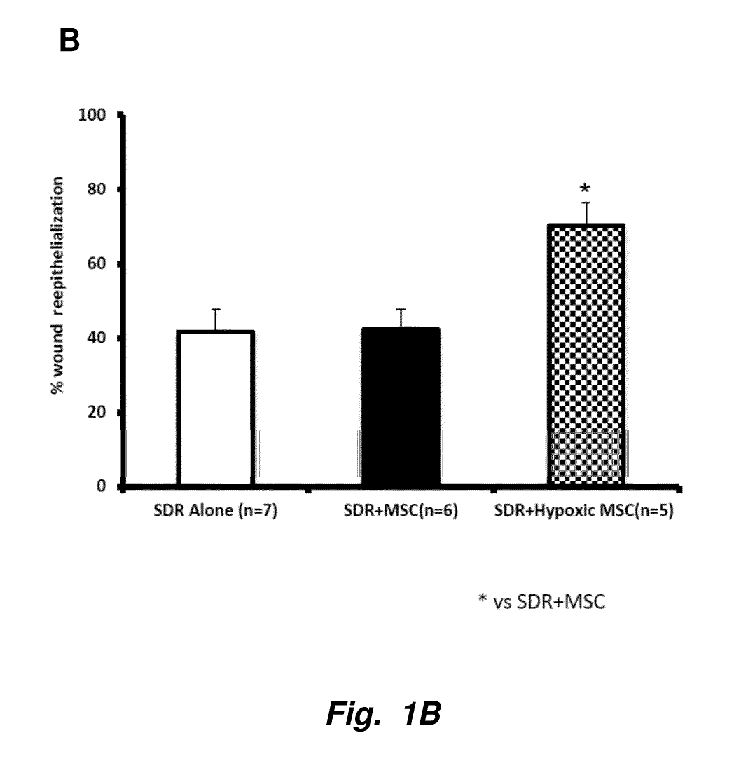

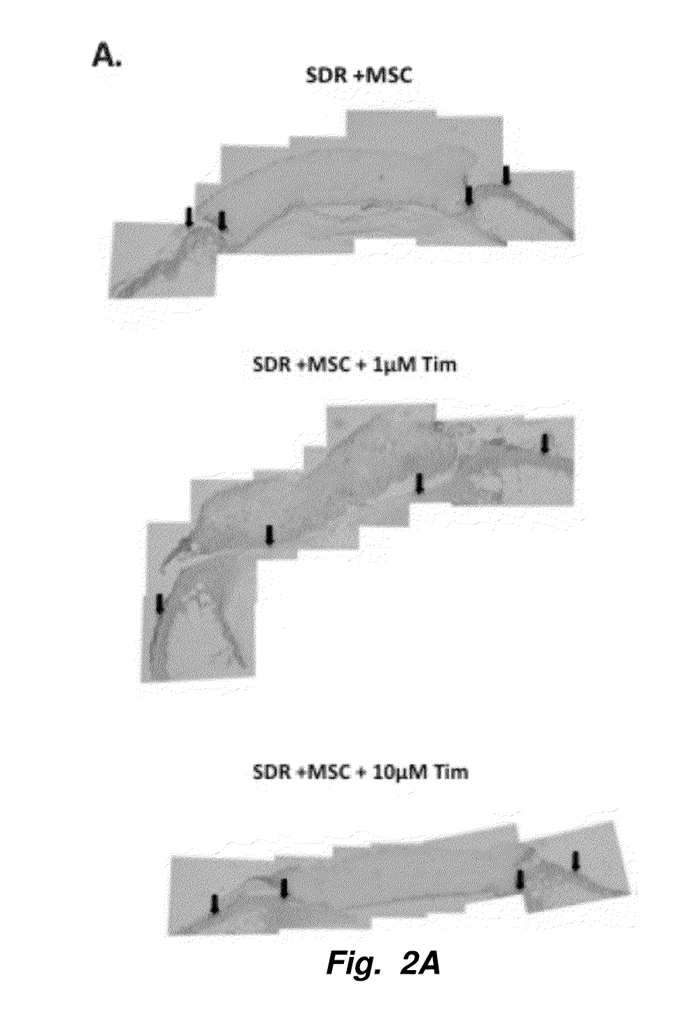

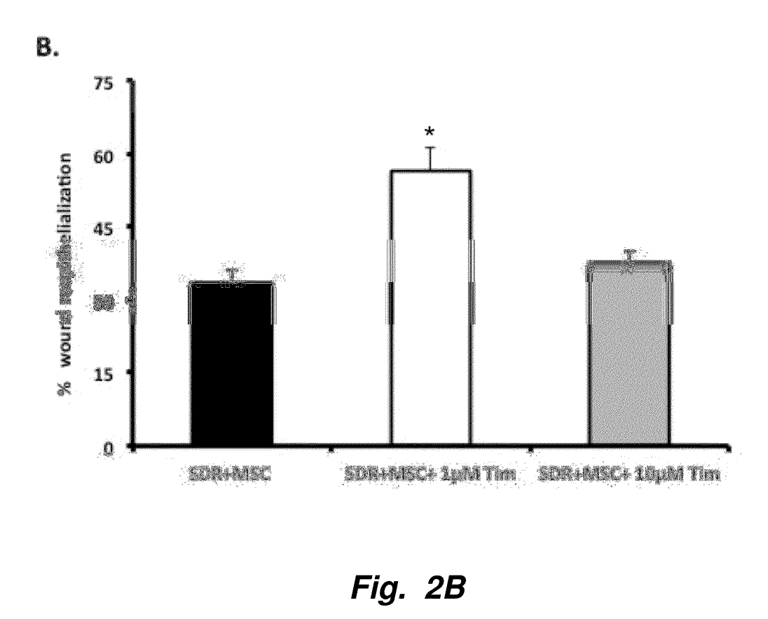

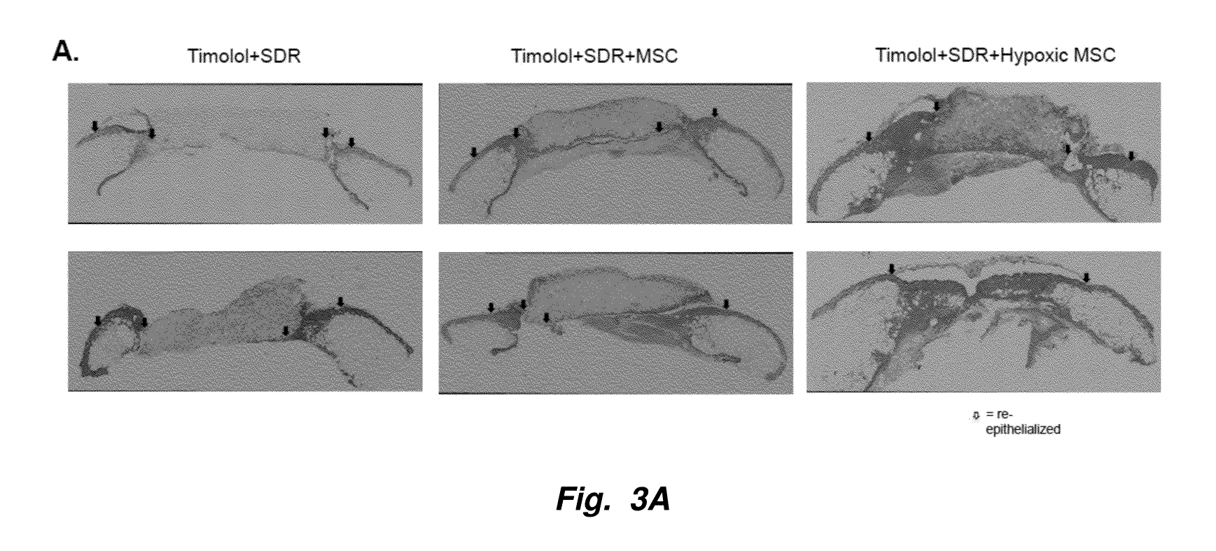

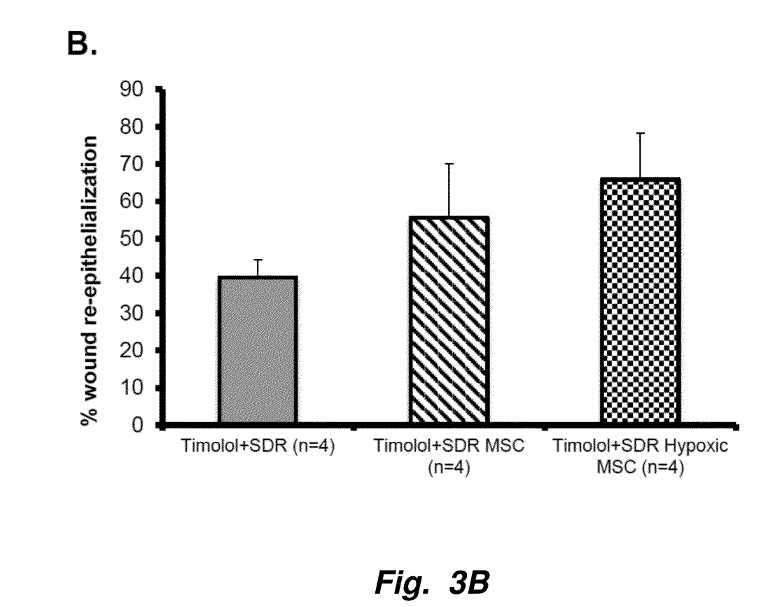

### DETAILED DESCRIPTION

### 1. Introduction

Provided are methods for the use of beta adrenergic receptor antagonist pre-conditioned human mesenchymal stem cells (MSC) embedded in an extracellular matrix Scaffold for Dermal Regeneration (SDR) for the treatment of epithelial and/or cutaneous wounds, e.g., diabetic wounds, venous and decubitus ulcers.

The present methods, compositions and kits are based, in part, on the surprising discovery that the combined use of a beta adrenergic receptor antagonist, MSCs, and SDR as an effective therapeutic device for the treatment of epithelial and/or cutaneous wounds, e.g., diabetic wounds. Herein we demonstrate that stress-induced inflammatory responses in full thickness cutaneous wounds of diabetic mice can be reduced by the addition of beta adrenergic receptor antagonists. Current topical therapies focus on the use of medications such as antibiotics, hyperbaric oxygen, all-trans retinoic acid, antimicrobials, among other treatments, but no treatments have utilized beta adrenergic receptor antagonists in conjunction with MSCs and SDR. Thus, we report the novel topical use of beta adrenergic receptor antagonist-conditioned MSC embedded in SDR for the treatment of epithelial and/or cutaneous wounds, e.g., diabetic wounds.

We have shown that inflammation plays a pivotal role in impaired wound healing in diabetic mice. Furthermore, we have shown that epinephrine can increase the release of inflammatory mediators and that use of a beta adrenergic receptor antagonist, such as timolol or ICI-118551, leads to suppression of the release of IL-6 by human keratinocytes. MSC are useful cellular therapy candidates for wound healing and commercially available extracellular matrix scaffolds for dermal regeneration (e.g., Integra) are currently in use for the treatment of burn and other wounds. Herein we demonstrate that beta antagonist-preconditioned MSC in SDR is useful for the suppression of the inflammatory response and improve healing. Further, a topical solution of a beta antagonist (timolol) can be applied to the MSC+SDR implanted, sutured or embedded in the diabetic wound (e.g., every other day or as appropriate) to continue the conditioning of the MSC with the beta adrenergic receptor antagonist.

Beta adrenergic receptor antagonists are widely used in medical practice both as systemic agents for cardiovascular disease and as topical agents for the eye. Illustrative beta adrenergic receptor antagonists of use include without limitation timolol and ICI-118551. Timolol is already in use as an FDA approved drug for use for glaucoma. Integra (SDR) is an FDA approved wound care device comprised of a porous matrix of cross-linked bovine tendon collagen and glycosaminoglycan. The collagen-glycosaminoglycan biodegradable matrix provides a scaffold for MSC retention. Preconditioning of MSC+SDR with a beta adrenergic receptor antagonist promotes and facilitates embedded or transplanted MSC survival and function better in the catecholamine rich wound microenvironment.

Current topical methods for treating DFW includes debridement to remove necrotic and infected tissues, dressings to provide a moist wound environment, bandages, and topical applications of antimicrobial or biologic agents, offloading, physical therapies, and educational strategies. However, these different treatment modalities often fail to achieve complete wound closure since they do not address the main culprit, i.e., persistent inflammation. For example, excessive use of antibiotics may address bacterial numbers and to some extent inflammation but can lead to the development of resistant strains. The present methods, compositions and kits address the inflammatory response without promoting or causing undesirable side effects like the development of antibiotic-resistant bacteria.

### 2. Subjects Who can Benefit

Subjects who can benefit generally have an epithelial and/or cutaneous wound. A wound in an epithelial tissue typically disrupts the continuity of the epithelial layer. For example, a wound in the skin typically disrupts (e.g., completely removes a section of) the epidermis, and, depending on the depth of the wound, can also remove part of the dermis. Healing of a wound in an epithelial tissue generally involves migration and/or proliferation of cells surrounding the wound, and the wound is typically considered to be healed when the wound is re-epithelialized, e.g., covered by at least one layer of cells.

In one aspect, the present extracellular matrices and methods provide for increasing the rate of repair, re-epithelialization and dermal regeneration of wounds in epithelial tissues, e.g., in humans. The methods involve the suturing, embedding and/or implanting of an extracellular matrix comprising embedded mesenchymal stem cells that have been exposed to, pre-conditioned with and/or cultured in the presence of beta adrenergic receptor antagonists to stimulate wound repair (i.e., re-epithelialization of the area), e.g., by stimulating migration and/or proliferation of epithelial cells (e.g., of keratinocytes for repair of a wound in the skin) and by decreasing the mediators of wound inflammation.

In varying embodiments, the target patient is a subject comprising or at risk for comprising a wound in an epithelial tissue. In varying embodiments, the wound is in skin. The methods and matrices described herein can be particularly useful for stimulating healing of chronic, non-healing skin wounds, which oftentimes are not sterile. In some embodiments, the wound comprises a chronic skin wound, e.g., a venous stasis ulcer, a diabetic foot ulcer, a neuropathic ulcer, or a decubitus ulcer. Other exemplary chronic wounds for which the methods can be used include, but are not limited to, other chronic ulcers such as immune-mediated (e.g., rheumatoid arthritis) ulcers, radiotherapy-induced ulcers, and ulcers resulting from vasculitis, arteriolar obstruction or occlusion, pyoderma gangrenosum, thalessemai, and other dermatologic diseases that result in non-healing wounds. In a related class of embodiments, the wound results from surgical wound dehiscence.

The methods and matrices described herein can also be applied to other types of wounds. For example, the wound can comprise a burn, cut, incision, laceration, ulceration, abrasion, or essentially any other wound in an epithelial tissue.

### 3. Beta Adrenergic Receptor Antagonists

A wide variety of beta-adrenergic receptor antagonists are known and have been described in the scientific and patent literature, many of which are in clinical use for other conditions. Although a few exemplary antagonists are listed below, no attempt is made to identify all possible agonists and antagonists herein. Other suitable antagonists which of use can be readily identified by one of skill in the art.

In varying embodiments, the beta adrenergic receptor antagonist is selective for the β2 adrenergic receptors, affecting or antagonizing substantially only the β2 adrenergic receptors. In some embodiments, the beta adrenergic receptor antagonist is nonselective, affecting or antagonizing the β1 and β2 adrenergic receptors, the β1, β2 and β3 adrenergic receptors, or the like. It will be evident that selectivity is optionally a function of the concentration of the antagonist. For example, an antagonist can have a Ki for the β2 adrenergic receptor that is 100-fold less than its Ki for the β1 adrenergic receptor, in which example the antagonist is considered to be selective for the β2 adrenergic receptor over the β1 adrenergic receptor when used at a concentration relatively near its Ki for the β2 adrenergic receptor (e.g., a concentration that is within about 10-fold of its Ki for the β2 adrenergic receptor).

In varying embodiments, the antagonist of a beta-adrenergic receptor is a non-selective antagonist for β1 and β2 adrenergic receptors. Illustrative non-selective antagonists of beta-adrenergic receptors include without limitation, e.g., carteolol, carvedilol, dilevelol, labetalol, nadolol, penbutolol, pindolol, propranolol, sotalol, timolol, and mixtures, analogs and salts thereof. In varying embodiments, the antagonist of a beta-adrenergic receptor is a selective antagonist for β1 adrenergic receptors. Illustrative selective antagonists for β1 adrenergic receptors include without limitation from the group consisting of acebutolol, atenolol, betaxolol, bisoprolol, celiprolol, esmolol, metoprolol, nebivolol, and mixtures, analogs and salts thereof. In varying embodiments, the antagonist of a beta-adrenergic receptor is a selective antagonist for β2 adrenergic receptors. Illustrative selective antagonists for β2 adrenergic receptors include without limitation ICI 118,551 and butoxamine.

In varying embodiments, the beta adrenergic receptor antagonist can be selective or nonselective for the β2 adrenergic receptors. Similarly, in certain embodiments, the antagonist has a greater affinity for the β2 adrenergic receptors than for the β3 adrenergic receptors. Thus, in one aspect, the antagonist has a Kd for a β3 adrenergic receptor that is about 100 or more times greater than a Kd of the antagonist for a β2 adrenergic receptor. In one aspect, the antagonist is substantially free of activity as a β3 adrenergic receptor agonist, e.g., has no detectable or significant activity as a β3 adrenergic receptor agonist. For example, in some embodiments, the scaffolds and methods optionally exclude CGP 12177.

The choice of antagonist for a particular application can be influenced, for example, by factors such as the half-life of the compound, its selectivity, potential side effects, preferred mode of administration, potency, and clinical information about a given patient (e.g., any known pre-existing conditions that might be exacerbated by administration of an agonist or antagonist, potential drug interactions, or the like). Nadolol has a long half-life (on the order of 24 hours), and potentially has lower central nervous system side effects due to low lipid solubility.

The concentration of beta adrenergic receptor antagonist cultured with the MSCs or amount of antagonist to be administered to the wound can depend on several factors, including without limitation, the nature, severity, and extent of the wound to be treated, the potency of the compound, the patient's weight, the patient's clinical history and response to the antagonist, and the discretion of the attending physician. Appropriate dosage can readily be determined by one of skill in the art. In varying embodiments, MSCs pre-conditioned with or exposed to beta-adrenergic receptor antagonists and embedded in an extracellular matrix or SDR are first implanted, embedded or sutured into or onto a wound, and then subsequent additional administrations of beta-adrenergic receptor antagonists are administered to the subject, e.g., either systemically administered or locally applied directly to the wound and the extracellular matrix within or on the wound.

Follow-up administrations of the beta adrenergic receptor antagonist can be administered to the patient at one time or over a series of administrations, as appropriate. For repeated administrations over several days or longer, depending on the condition, the treatment is optionally sustained until a desired result occurs; for example, until a wound is healed. Similarly, treatment can be maintained as required. The progress of the therapy can be monitored by conventional techniques and assays.

The antagonist can be administered systemically, locally, and/or topically. For example, the antagonist can be administered systemically, e.g., orally or intravenously. As another example, the antagonist can be administered topically, e.g., by application of an ointment, cream, lotion, gel, suspension, spray, dressing, transdermal device, foam, or the like comprising the antagonist to the wound. As yet another example, the antagonist can be administered locally or intralesionally by injecting the antagonist directly into tissue underlying or immediately adjacent to the wound. For example, for a skin wound, the antagonist can be administered by injecting it subcutaneously or intradermally at or near the site of the skin wound. In varying embodiments, the beta adrenergic receptor antagonist is directly attached to the extracellular scaffold matrix, e.g., via covalent bonding or crosslinking. Crosslinkers of use are known in the art. In varying embodiments, the beta adrenergic receptor antagonist is directly attached or crosslinked to the extracellular scaffold matrix using a linkage chemistry or integrated biodegradable matrix (e.g., Poly(D,L-lactide-co-glycolide (PLGA) beads).

A pharmaceutical composition for topical administration of a beta adrenergic receptor antagonist, e.g., an ointment, cream, lotion, foam, or gel (e.g., an aqueous gel), or, in general, a solution or suspension of the agonist or antagonist, typically contains from 0.01 to 10% w/v (weight/volume, where 1 g/100 ml is equivalent to 1%) of the agonist or antagonist, preferably from 0.1 to 5% w/v, e.g., mixed with customary excipients or dissolved in an appropriate vehicle for topical application. Exemplary compositions formulated for topical application to skin can comprise an ointment (e.g., an occlusive or petrolatum-based ointment), cream, lotion, gel, spray, foam, or the like, e.g., in which the antagonist is suspended, dissolved, or dispersed. Many suitable bases for such ointments, creams, lotions, gels, etc. are known in the art and can be used. At least one component of the composition is optionally insoluble in water and/or hydrophobic; for example, the composition optionally includes an oil (e.g., a suspension of an oil in water), petrolatum, a lipid, or the like.

In a pharmaceutical composition for oral, sublingual, subcutaneous, intramuscular, intravenous, transdermal, or local administration, for example, the agonists or antagonists can be administered in unit forms of administration, either as such, for example in lyophilized form, or mixed with conventional pharmaceutical carriers. Appropriate unit forms of administration include oral forms such as tablets, which may be divisible, gelatin capsules, powders, granules and solutions or suspensions to be taken orally, sublingual and buccal forms of administration, subcutaneous, intramuscular or intravenous forms of administration, and local forms of administration.

When a solid composition is prepared in the form of tablets, the main active ingredient is optionally mixed with a pharmaceutical vehicle such as gelatin, starch, lactose, magnesium stearate, talcum, gum arabic or the like. The tablets can be coated with sucrose or other appropriate substances, or can be treated so as to have a prolonged or delayed activity and so as to release a predetermined amount of active principle continuously. A preparation in the form of gelatin capsules can be obtained by mixing the active ingredient with a diluent and pouring the resulting mixture into, soft or hard gelatin capsules. A preparation in the form of a syrup or elixir optionally contains the active ingredient together with a sweetener, antiseptic, flavoring and/or appropriate color. Water-dispersible powders or granules can contain the active ingredient mixed with dispersants, wetting agents or suspending agents, as well as with sweeteners or taste correctors. Suppositories (e.g., for vaginal or rectal administration) can be prepared with binders melting at the appropriate (e.g., vaginal or rectal) temperature. Parenteral administration is typically effected using aqueous suspensions, saline solutions or injectable sterile solutions containing pharmacologically compatible dispersants and/or wetting agents. The antagonist is optionally encapsulated in liposomes or otherwise formulated for prolonged or delayed release, e.g., whether for topical, local, and/or systemic administration.

In varying embodiments, the MSCs are exposed to a concentration of beta adrenergic receptor antagonist sufficient to supersede or overcome signaling through Toll-Like receptors (e.g., TLR2) facilitating the concomitant prevention, reduction and/or inhibition of the production of IL-6 and other inflammatory mediators that inhibit wound healing. In varying embodiments, the MSCs are exposed to, cultured in or preconditioned with a concentration of at least about 0.1 μM to about 50 μM beta adrenergic receptor antagonist, e.g., from at least about 1.0 μM to about 25 μM beta adrenergic receptor antagonist, e.g., from at least about 1.0 μM to about 10 μM beta adrenergic receptor antagonist. In varying embodiments, the MSCs are exposed to, cultured in or preconditioned with a concentration of at least about 0.2 μM to about 50 μM, e.g., about 0.4 μM to about 40 μM, e.g., about 0.3 μM to about 30 μM, e.g., about 0.2 μM to about 20 μM, e.g., about 1.0 μM to about 10 μM. In varying embodiments, MSCs within the extracellular matrix may have continued exposure to the beta adrenergic receptor antagonist. In varying embodiments, the beta adrenergic receptor antagonist is added to the matrix or culture medium one or multiple times, as needed to promote, facilitate and/or accelerate wound healing.

### 4. Mesenchymal Stem Cells

The methods and extracellular matrices described herein entail the administration to a wound of MSCs that have been contacted and/or pre-conditioned with and/or exposed to a beta adrenergic receptor antagonist. Data provided herein and in Dasu, et al., Stem Cells Transl Med. (2014) 3(6):745-59 (hereby incorporated herein by reference in its entirety for all purposes) demonstrate that exposing MSCs to a beta adrenergic receptor antagonist promotes and/or facilitates wound healing.

The bone marrow of an adult mammal is a repository of mesenchymal stem cells (MSCs). These cells are self-renewing, clonal precursors of non-hematopoietic tissues. MSCs for use in the present methods can be isolated from a variety of tissues, including bone marrow, muscle, fat (i.e., adipose), liver and dermis, using techniques known in the art. Illustrative techniques are described herein and reported in, e.g., Chung, et al., Res Vet Sci. (2010) November 12, PMID:21075407; Toupadakis, et al., American Journal of Veterinary Research (2010) 71(10):1237-1245.

Generally, the MSCs useful for administration express on their cell surface CD44 and CD90 and do not express on their cell surface CD34, CD45, CD80, CD86 or MHC-II. In various embodiments, the MSCs are adipose-derived mesenchymal stem cells (Ad-MSC). Ad-MSCs can be characterized by the surface expression of CD44, CD5, and CD90 (Thy-1); and by the non-expression of CD34, CD45, MHC class II, CD3, CD80, CD86, CD 18 and CD49d. In other embodiments, the MSCs are derived from a non-adipose tissue, for example, bone marrow, liver, lacrimal gland, and/or dermis. In some embodiments, the MSCs are non-haematopoietic stem cells derived from bone marrow (i.e., do not express CD34 or CD45).

As appropriate, the MSCs can be autologous (i.e., from the same subject), syngeneic (i.e., from a subject having an identical or closely similar genetic makeup); allogeneic (i.e., from a subject of the same species) or xenogeneic to the subject (i.e., from a subject of a different species).

In various embodiments, the MSCs may be altered to enhance the viability of the embedded, engrafted or transplanted cells. For example, the MSCs can be engineered to overexpress or to constitutively express Akt. See, e.g., U.S. Patent Publication No. 2011/0091430.

### 5. Extracellular Matrix Scaffold

In various embodiments, embedding, engraftment or transplantation of the beta adrenergic receptor antagonist-conditioned MSCs is facilitated using a matrix or caged depot, e.g., an extracellular matrix scaffold. For example, the MSCs can be embedded, engrafted or transplanted in a “caged cell” delivery device wherein the cells are integrated into a biocompatible and/or biologically inert matrix (e.g. a hydrogel or other polymer or any device) that restricts cell movement while allowing the cells to remain viable. Synthetic extracellular matrix and other biocompatible vehicles for delivery, retention, growth, and differentiation of stem cells are known in the art and find use in the present methods. See, e.g., Prestwich, J Control Release. 2011 Apr. 14, PMID 21513749; Perale, et al., Int J Artif Organs. (2011) 34(3):295-303; Suri, et al., Tissue Eng Part A. (2010) 16(5):1703-16; Khetan, et al., J Vis Exp. (2009) October 26; (32). pii: 1590; Salinas, et al., J Dent Res. (2009) 88(8):681-92; Schmidt, et al., J Biomed Mater Res A. (2008) 87(4):1113-22 and Xin, et al., Biomaterials (2007) 28:316-325. The extracellular matrix can be naturally occurring (e.g., decellularized tissue) or synthetic.

Any biocompatible, biodegradable matrices known in the art can be used as a scaffold or extracellular matrix for the MSCs. In varying embodiments, the matrix is made of naturally derived components (e.g., collagen, elastin, laminin, gelatin and/or other naturally derived materials). In varying embodiments, the matrix can be synthetic or made of or comprise non-naturally derived components. Biocompatible, biodegradable materials useful in the matrices include, e.g., polyglycolic acid (PGA), type 1 collagen, Poly-DL-lactide-caprolactone (PCL), laminin, gelatin, chitin, alginate, keratin, and the like. In varying embodiments, the matrix comprises collagen. Illustrative extracellular matrices that are commercially available and find use include without limitation, e.g., matrices available from Integra Life Sciences (integralife.com); Oasis Wound matrices available from Cook Biotech (oasiswoundmatrix.com); MatriStem matrices from ACell (acell.com); GRAFTJACKET® matrices from Wright Medical Technology (wmt.com); MatriDerm® matrices by MedSkin Solutions Dr. Suwelack AG (medskin-suwelack.com); and UNITE™ Biomatrices by Baxter Healthcare (synovissurgical.com).

As appropriate or desired, the embedded, engrafted or transplanted beta adrenergic receptor antagonist-conditioned MSCs can be modified to facilitate retention of the MSCs at the region of interest or the region of delivery, e.g., at the site of the wound. In other embodiments, the region of interest for embedding, engraftment or transplantation of the cells is modified in order to facilitate retention of the MSCs at the region of interest or the region of delivery. In one embodiment, this can be accomplished by introducing stromal cell derived factor-1 (SDF-1) into the region of interest, e.g., using a linkage chemistry or integrated biodegradable matrix (e.g., Poly(D,L-lactide-co-glycolide (PLGA) beads) that would provide a tunable temporal presence of the desired ligand up to several weeks. MSCs bind to the immobilized SDF-1, thereby facilitating the retention of MSCs that are delivered to the region of interest for embedding, engraftment or transplantation. In other embodiments, integrating cyclic arginine-glycine-aspartic acid peptide into the region of interest can facilitate increased MSC binding and retention at the region of interest for embedding, engraftment or transplantation. See, e.g., Ratliff, et al., Am J Pathol. (2010) 177(2):873-83.

In varying embodiments, at least about 0.25×10⁶ MSCs are provided to the subject, e.g., in the matrix embedded, engrafted or implanted at the site of the wound. As appropriate, the number of MSCs injected into the subject or embedded, engrafted or implanted into the matrix at the site of the wound can be at least about, e.g., 1×10⁴ cells, 2.5×10⁴ cells, 5×10⁴ cells, 7.5×10⁴ cells, 1×10⁵ cells, 2.5×10⁵ cells, 5×10⁵ cells, 7.5×10⁵ cells, 1×10⁶ cells, 2.5×10⁶ cells, 5×10⁶ cells, 7.5×10⁶ cells, 1×10⁷ cells, 2.5×10⁷ cells, 5×10⁷ cells, 7.5×10⁷ cells, or 1×10⁸ cells.

In various embodiments, the cells can be delivered or embedded in the extracellular matrix at a concentration in the range of about 1×10⁶ cells/ml to about 1×10⁸ cells/ml, for example, in the range of about 5×10⁶ cells/ml to about 5×10⁷ cells/ml, for example about 1×10⁶ cells/ml, 5×10⁶ cells/ml, 1×10⁷ cells/ml, 5×10⁷ cells/ml or 1×10⁸ cells/ml.

The total amount of cells that are envisioned for use depend upon the desired effect, patient state, and the like, and may be determined by one skilled within the art. Dosages for any one patient depends upon many factors, including the patient's species, size, body surface area, age, the particular MSCs to be administered, sex, scheduling and route of administration, general health, and other drugs being administered concurrently.

### EXAMPLES

The following examples are offered to illustrate, but not to limit the claimed invention.

### Example 1

### Cross Talk Between Adrenergic and Toll-Like Receptors in Human Mesenchymal Stem Cells and Keratinocytes

### Methods

Mesenchymal Stem Cells (MSC).

Human bone marrow aspirations obtained from four healthy donors were purchased from Lonza. MSC were harvested from bone marrow (BM) following established protocols (26, 27), and used between passages 3-5. Characterization of MSC included differentiation into osteogenic and adipogenic lineage cells, as described previously (27). The stem cell research oversight (SCRO) review board at the University of California, Davis approved all the human cell protocols.

Neonatal Human Keratinocytes (NHK).

NHK were isolated from human neonatal foreskins, cultured and maintained, as reported earlier (28,29). NHKs isolated from at least three different foreskins and between passages 3 and 7 were used in all the experiments. The Institutional Review Board at UC Davis approved the protocol for obtaining discarded neonatal foreskins.

Cell treatments.

Epinephrine (EPI: Sigma) and TLR2 ligands (macrophage activating lipoprotein-2: MALP2—that specifically activates TLR2/6 heterodimerization, and heat killed Staphylococcus aureus: HKSA; Invivogen) treatments were carried out at the indicated times and concentrations. All the cells were maintained in 0.5% fetal bovine serum containing culture medium overnight prior to treatment. Cells were exposed to different treatments in fresh serum-free medium. In some experiments, cells were pretreated for 30 minutes with Timolol (10 μM; Sigma) or ICI-118, 551 (ICI; 10 μM; Tocris) followed by EPI and MALP2 treatment as described previously (11-13, 30).

Single-Cell Migration.

NHK and BM-MSC were plated on collagen I-coated plates as reported previously (11-13,30). Time-lapse images of the cell migration were captured every 5 minutes for 1 hour. The distance cells travel in a one-hour time period is recorded and indicated as the average speed (μm per minute). Significance was set at P<0.05, and Student's t test (unpaired) was used to compare the means of two cell populations as reported previously (11-13, 30).

Animals with EPI Osmotic Pumps and Full-Thickness Cutaneous Wounds.

C57BL/6J (male; 8-10 week age; Jax Mice) with ad libitum access to food and water were anesthetized using isoflurane and one 6 mm circular diameter full-thickness wound was placed on the dorsal shaved skin β1). Micro-osmotic pumps (0.25 μl/hour; Alzet micro-osmotic pump Model: 1002, Alzet) were implanted on the right flank of the mice to deliver 7 mg/kg body weight/day EPI and 0.7 mg/kg body weight/day of ICI) as we have previously reported (11-13,30). At 7 or 11 days after injury, the mice were euthanized, the wound tissue was harvested by 8 mm punch excision and stored frozen or formalin—fixed until further analysis. Animal protocols were approved by the IACUC at UC Davis.

Real Time PCR (RT-PCR).

mRNA expression was determined by real-time PCR (RT-PCR), using sequence-specific primers and probes. Total RNA was extracted from the cells using Qiagen RNeasy mini kit. The first strand of cDNA was synthesized using 1 μg of total RNA. cDNA (50 ng) was amplified using primer probe sets for TLR2, beta-2-adrenergic receptor (ADRB2) and three housekeeping genes: beta-2-microglobulin, GAPDH, and RPLPO using standard cycling parameters. Data were calculated using the 2-ΔΔCt method and are presented as fold change (ratio of transcripts of gene normalized to the three house-keeping genes) (11-13, 31).

ELISA.

Levels of interleukin-6 (IL-6) were measured with an ELISA kit (R&D systems). IL-6 levels were normalized to total cell protein and expressed as pg/μg protein β1).

Western Blots.

Twenty five μg of total protein was resolved, transferred, and probed with antibodies for β2-AR (Abcam), phospho beta-adrenergic receptor activated kinase-1 (BARK-1/GRK2 referred to as BARK-1 from here after; GeneTex), TLR2 (Imgenex) Myeloid differentiation factor 88 (MyD88; Imgenex), phospho interleukin receptor activated kinase-1 (pIRAK-1 and IRAK-1; Cell Signaling), phospho ERK1/2 (Santacruz), phenylethanolamine N-methyltransferase (PNMT) and tyrosine hydroxylase (TH), and stripped membranes were further incubated with respective total antibodies or GAPDH or α-tubulin. TLR2 (Imgenex) and β2-AR (Santacruz) antibodies were used for co-immunoprecipation assays and antibody protein complexes were further probed with above antibodies β1). Protein A/G-sepharose beads and isotype matched IgG antibodies were used as negative controls in all the co-immunoprecipitation experiments along with the antibody used for the pull down as a positive control. Band intensities were determined as described previously and normalized to GAPDH/α-tubulin or total protein (BARK-1) and densitometric ratios are presented as fold change vs control β1). For some experiments, cell lysates from three independent experiments were pooled to get enough protein for the assay and repeated three times for densitometry purposes.

Scratch Wound Assays.

The rate of healing scratch wounds made in confluent NHK cultures was determined as reported previously (30, 32). Briefly, cells were pretreated with 10 μg/ml mitomycin (EMD Millipore) for 1 hr to inhibit cell proliferation that could skew the data analysis. Wounded cultures were incubated in growth medium (control) containing EPI, TLR2 ligands, and/or Timolol or ICI. Velocity Image analysis software (Perkin Elmer) was used to measure the scratch wound area, which is expressed as percent closed wound.

HPLC Detection of Catecholamines.

Cell culture samples from at least from three independent experiments were acidified with perchloric acid to 0.2N prior to storage at −80° C. for future analysis. The supernatant were applied to conditioned MonoSpin® PBA solid phase extraction spin columns (GL Sciences) and purifications were performed according to the manufacturer's specifications. Catecholamines were eluted in 200 μL of 2% acetic acid.

HPLC separation was performed using a Synergi™ 4 um Fusion-reverse phase 250×4.6 mm column (Phenomenex) and a HP series 1050 pump and auto injector system. The mobile phase for chromatographic separation was a modification of that used by Leis et al (33). Detection of catecholamine compounds was performed using a LC-4C amperometric detector (Bioanalytical Systems) using potential of −700 mV. Catecholamine levels are presented as pg/μg cell protein.

Statistical Analysis.

Statistical analyses were performed using Excel and Graphpad Prism. Data are expressed as mean±S.D. Parametric data were analyzed using paired, two tailed t-tests and non-parametric data using Wilcoxon signed rank tests. Level of significance was set at P<0.05 (11-13, 30, 31).

### Results

1. EPI Induces TLR2 Expression and Signaling; Conversely TLR2/6-Specific Ligand MALP2 Upregulates β2-AR mRNA and Protein Expression in BM-MSC.

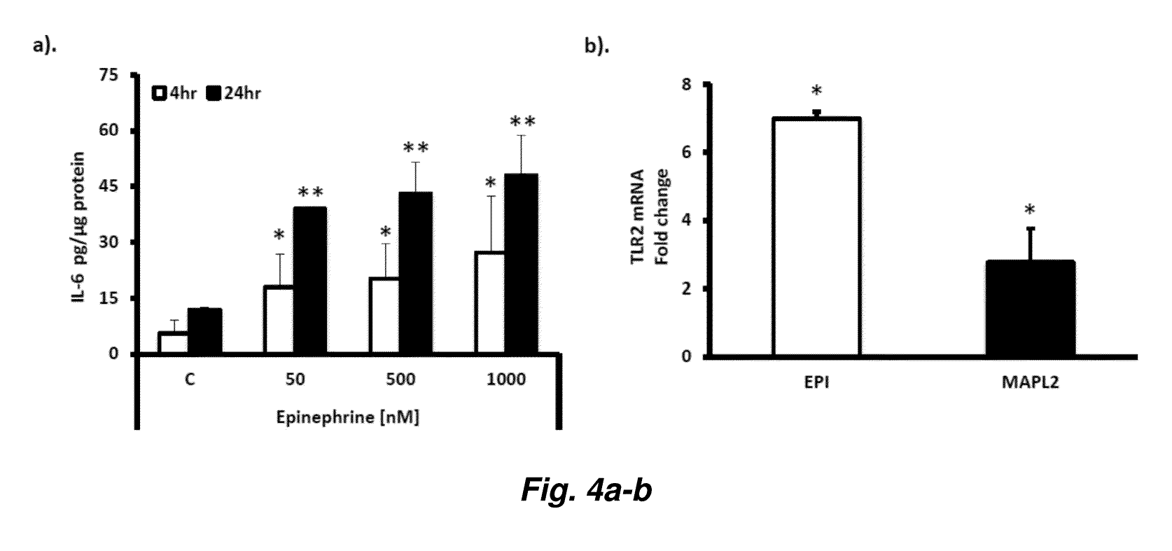

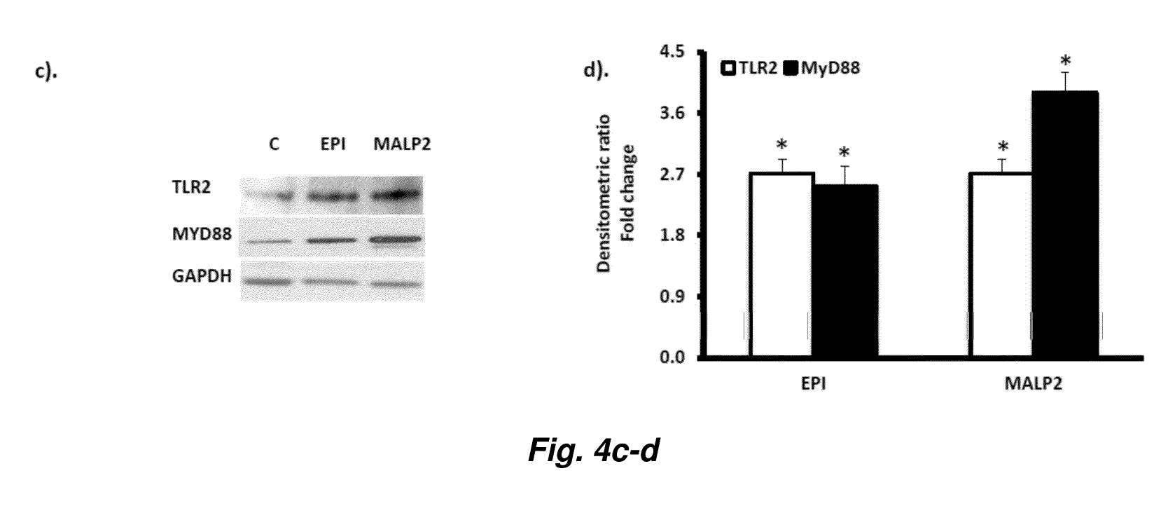

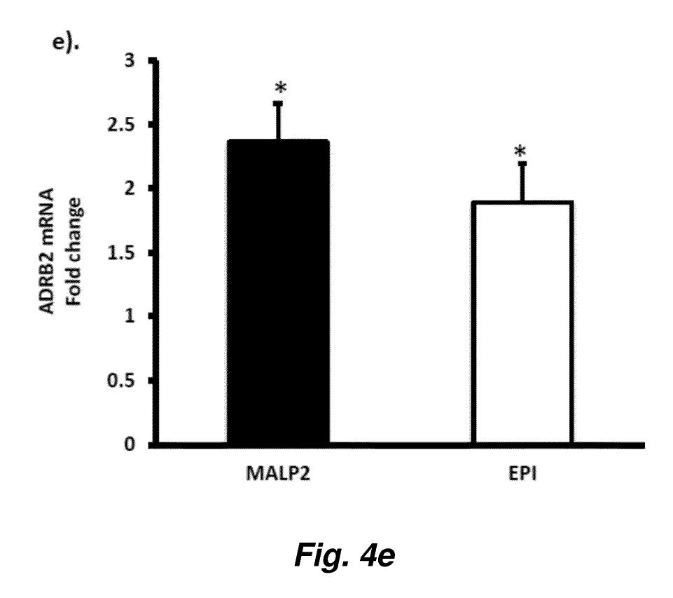

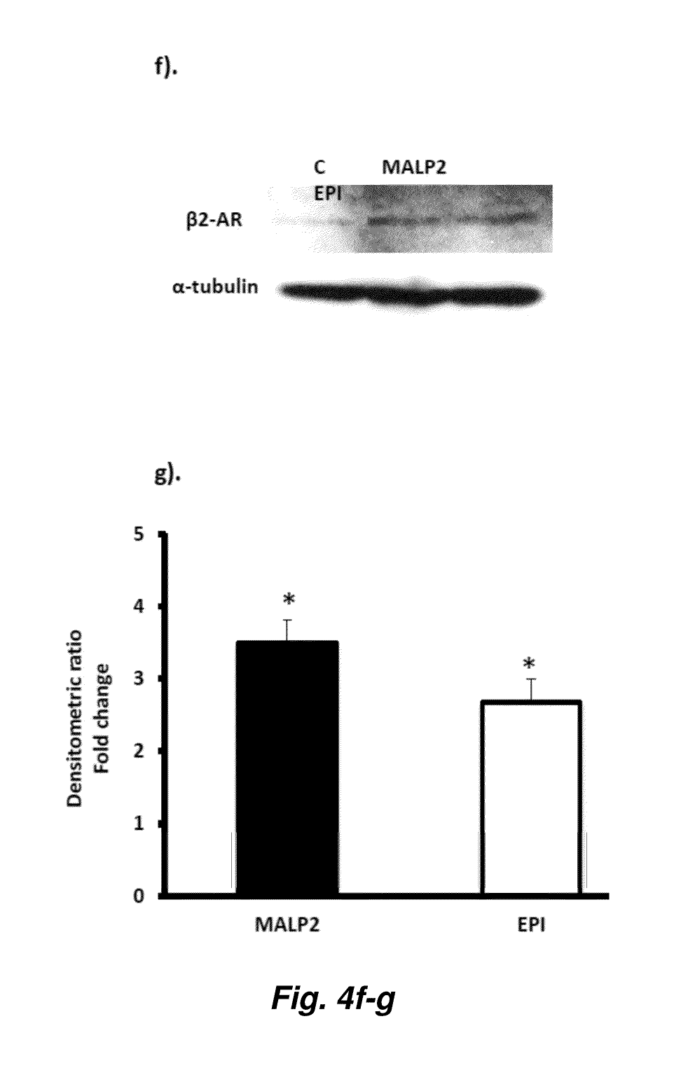

To address the question of how EPI stress impacts upon innate immune capabilities of BM-MSC, we examined the effect of EPI treatment on TLR2 expression and IL-6 secretion. EPI significantly induced IL-6 secretion in BM-MSC with maximal induction at 50 nM (FIG. 4a). The percent increase in secreted IL-6 did not vary between 4 hr and 24 hr; therefore, we selected 50 nM EPI (closer to stress levels in humans) (34) and 4 hrs duration (due to short half-life of EPI) for subsequent studies.

EPI significantly increased TLR2 expression in BM-MSC, both at the mRNA (FIG. 4b), and protein levels (FIG. 4c & d). MALP2 (a specific natural ligand; 35-37) activates TLR2 and initiates a signaling cascade by recruiting the adaptor protein MyD88 and ending with the expression of pro-inflammatory genes, like IL-6 (14, 36, 37). Indeed, we found that MyD88 protein expression is significantly elevated in EPI-treated BM-MSC (FIG. 4c), surprisingly, as robustly as when MALP2 was used to directly activate TLR2 (FIG. 4c & d). Presence of endotoxin in the cell culture medium was ruled out by pretreatment of the BM-MSC with Polymyxin B (widely used for neutralizing endotoxin effects; 38). Pretreatment with polymyxin B did not affect EPI or MALP2 induced IL-6 levels in the cells.

The question of whether TLR2 signaling could impact upon the β2-AR system is one we explored here. We examined whether the TLR2 agonist MALP2 could modulate the β2-adrenergic signaling cascade. Both mRNA and protein expression for the β2-adrenergic receptor are significantly up-regulated in BM-MSC treated with MALP2 (FIGS. 4e, f, & g). Of note, MALP2 is as effective as the β2-AR native ligand EPI in up regulating the receptor expression.

### 2. MALP2 Activation of TLR2 Leads to Synergistic β2-AR Expression and BARK-1 Phosphorylation in BM-MSC.

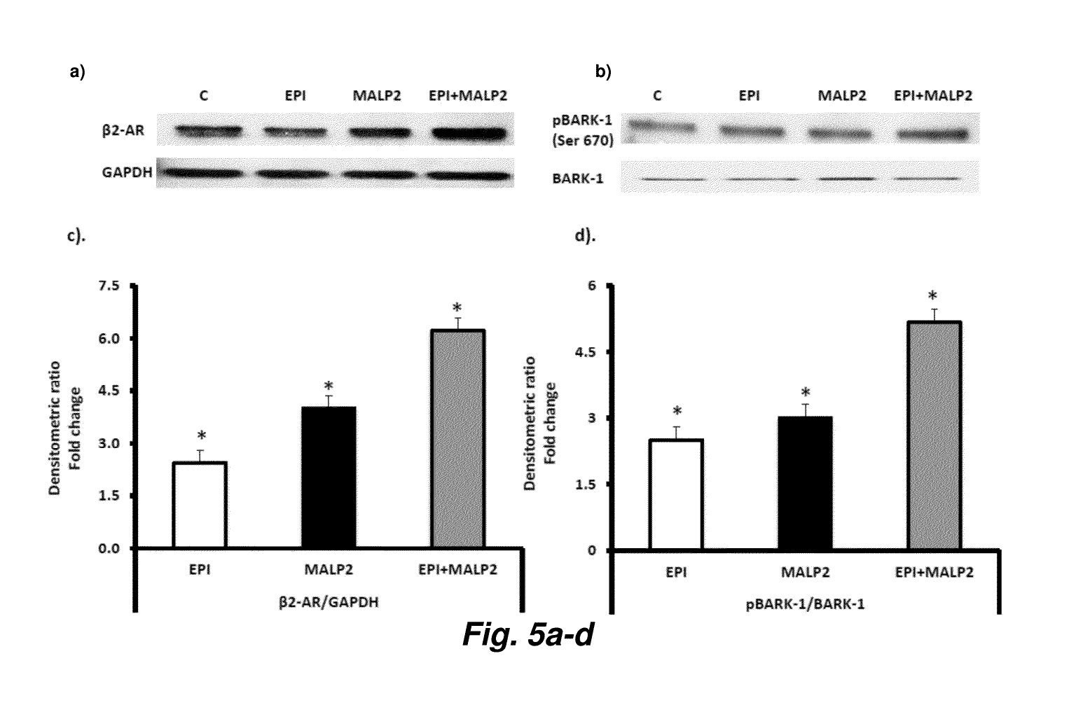

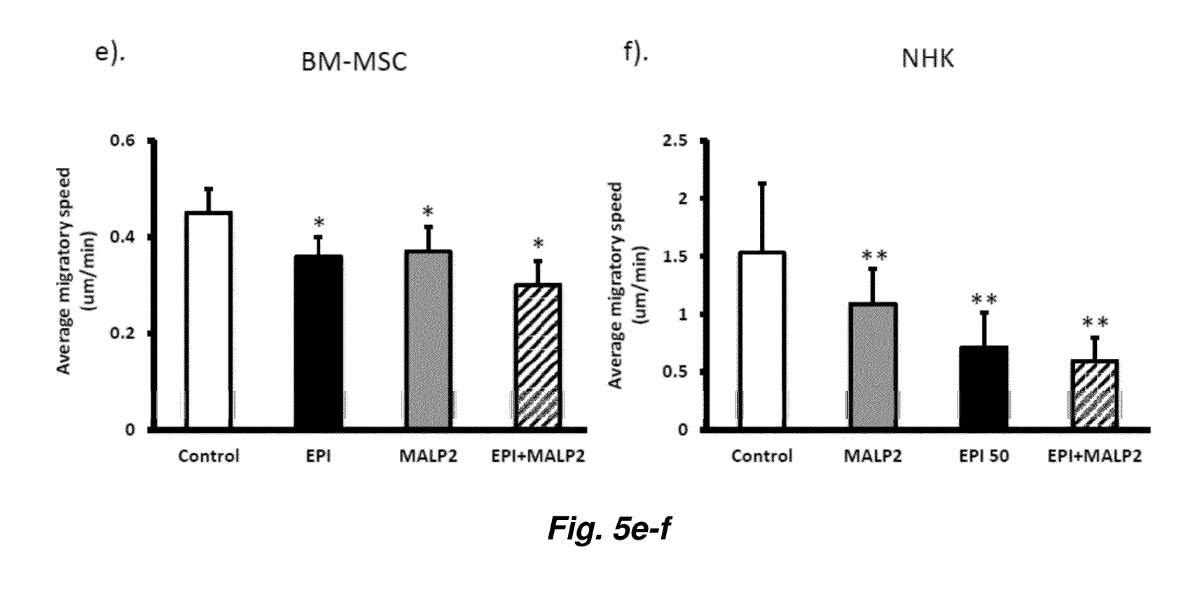

In murine macrophages, specific TLR ligands have been shown to increase BARK-1 protein expression (39), a downstream event generally ascribed to β2-AR stimulation (40-42). To determine if MALP2 activation of the TLR2 could also result in synergistic downstream signaling through the β2-AR pathway, we examined the phosphorylation of BARK-1 (39). MALP2 stimulation of BM-MSC significantly increased BARK-1 phosphorylation (FIG. 5a, b) indicating that MALP2 may be able to initiate signaling downstream to β2-AR, and as potently as the receptor's cognate ligand, EPI. Activation of both the β2-AR and TLR2 pathways simultaneously, by incubation of BM-MSC with EPI and MALP2 resulted in a synergistic increase in both β2-AR receptor expression by 6 fold and phosphorylation of BARK-1 by five-fold (FIG. 5c,d).

### 3. EPI and MALP2 Inhibit Migration of BM-MSC and NHK

Migration of MSC to the wound site (18, 20, 21) and NHK migration for wound re-epithelialization (22) are both critical for optimal healing. Therefore, we addressed the question of how β2-AR and TLR cross-talking pathways could affect migration in these two cell types. EPI treatment reduced BM-MSC migratory speed by 10% (FIG. 5e), as we have previously reported (43, 44). Activation of the TLR2 receptor with MALP2 (100 ng/ml) resulted in a similar decrease (15%) in migratory speed (FIG. 5e). The combined activation of β2-AR and TLR2 resulted in a 21% inhibition in migratory speed. Similar impairment of NHK migration was observed (FIG. 5f) with activation of either the TLR2 or β2-AR, except that the effects were several folds higher than in BM-MSC (FIG. 5d). Combined treatment of NHK with both EPI and MALP2 resulted in 60% inhibition of the migratory speed (FIG. 5f). We did not observe appreciable differences in migration between 1, 4, and 24 hr analyses of cell migration. Both treated and untreated cells showed about 95% viability (as determined by MTS assay) with minimal cytotoxicity (as determined by LDH activity). These results suggest that in a wound environment, activation of stress and innate immune receptors together could adversely affect cell migration that is critical for the healing process.

### 4. EPI and MALP2/HKSA Stimulate IL-6 Secretion in BM-MSC and NHK

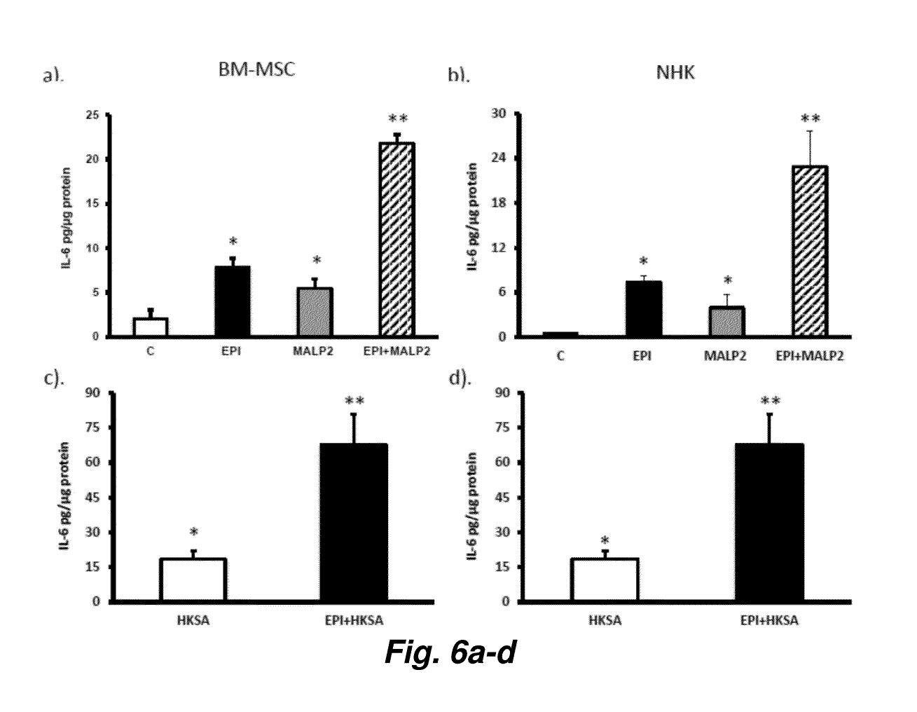

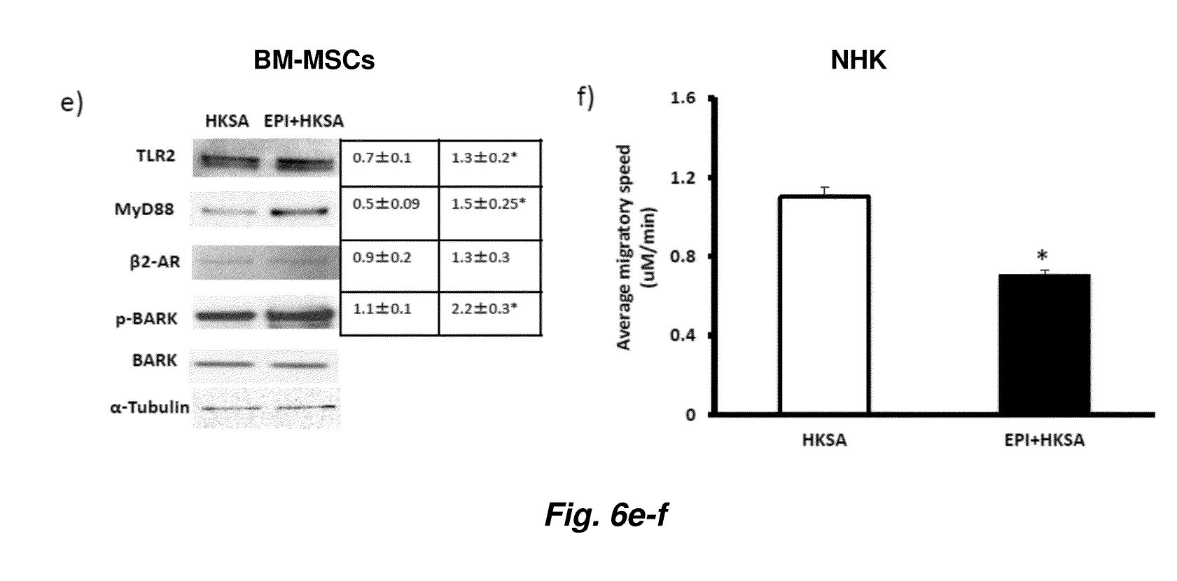

We investigated the downstream convergence of these two distinct receptor-signaling systems by measuring the release of the inflammatory cytokine, IL-6 from cells treated with EPI or MALP2. Both agents induced significant increases (3.8 and 2.6 fold, respectively) in IL-6 secretion in BM-MSC as compared to untreated control cells (FIG. 6a), and treatment with a combination of the two agents increased IL-6 expression in BM-MSC to >10 fold over control (FIG. 6a). These effects are more pronounced in NHK, where EPI and MALP2 alone induced 16 fold and 8.8 fold increase in IL-6 secretion respectively, as compared to untreated NHK (FIG. 6b), and combining the two agents resulted in a 51-fold increase in IL-6 release relative to control (FIG. 6b). To complement the MALP2 studies, we used HKSA (10⁴ cells/ml) that also ligates the TLR2 (45). HKSA increased IL-6 secretion in BM-MSC and NHK by 3-fold, and this was 13 and 4-fold increased upon addition of EPI, compared to control (FIGS. 6c & 6d). Next, we asked whether the observed synergistic IL-6 increase induced by β2-AR and HKSA co-stimulation is coupled through TLR2-MyD88 and β2-AR-BARK-1 signaling. There was a significant increase in TLR2-MyD88 and β2-AR-pBARK-1 activation in BM-MSC (>2 fold change) treated with EPI and HKSA compared to HKSA alone (FIG. 6e). We also determined HKSA effects on cell migration with EPI. HKSA induced significant inhibition in NHK migration (16% inhibition; P<0.001) and this was further reduced in the presence of EPI (52% inhibition; FIG. 6f). These results indicate that EPI and MALP2/HKSA, through activation of their respective receptors, synergistically enhance down-stream signaling, inhibit cell migration, and stimulate IL-6 release in BM-MSC and NHK. Since the wound environment is likely to have elevated levels of both EPI (13) and bacterially-derived activators of the TLR2 receptor (46-48), this combination may negatively impact on healing by the synergism in generation of pro-inflammatory mediators such as IL6.

### 5. β2-AR Antagonists Reverse EPI and MALP2/HKSA Induced Changes in Cells

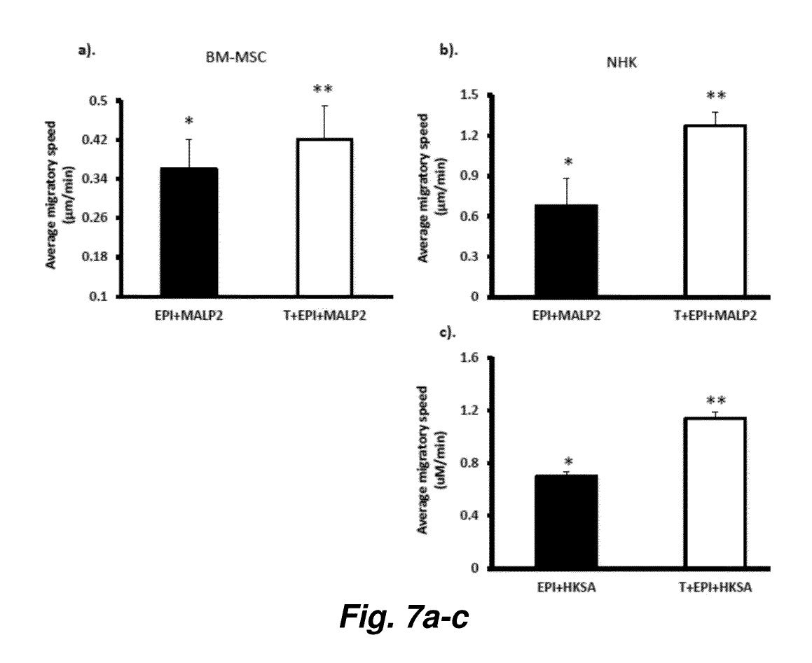

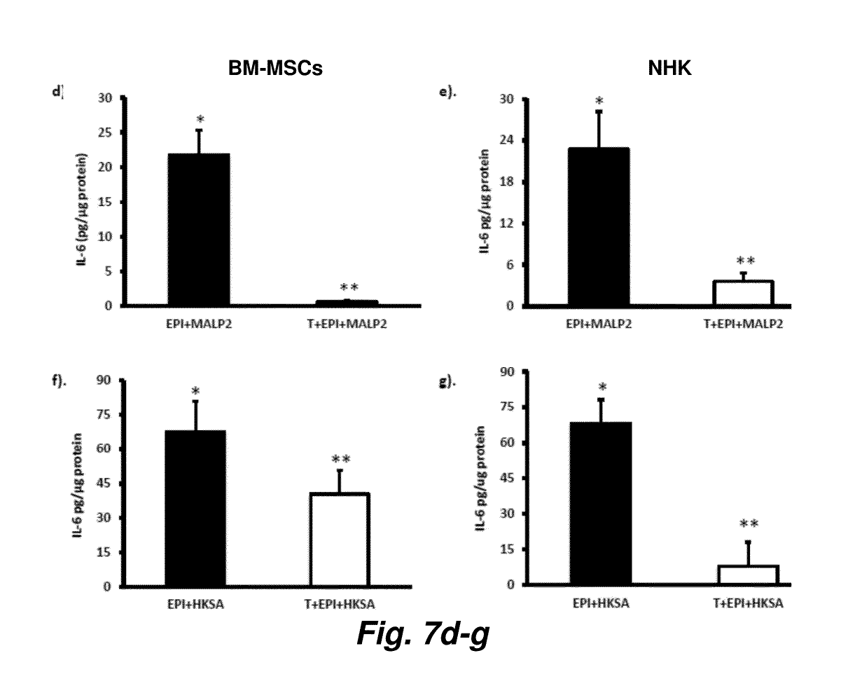

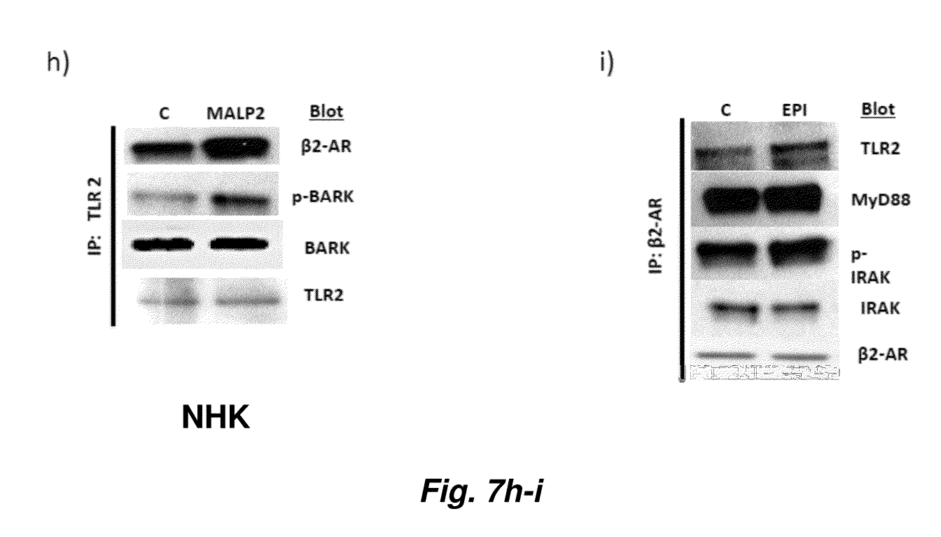

Next, we asked the question of whether blocking one of the receptors (β2-AR) could effectively increase cell migration and decrease IL-6, both critical for improved healing. β-blockers are being used clinically to improve outcomes in burn wound patients (49,50), and improve healing in chronic wounds (51,52). We selected the β1/2-AR antagonist, Timolol, that has been shown to reverse β2-AR inhibition of keratinocyte migration (32) and is a currently FDA approved drug. Pretreatment of cells with Timolol (10 μM, 30 min) reversed the EPI+MALP2 or EPI+HKSA synergistic effects on the cell migration and IL-6 release in BM-MSC and NHK. Timolol pretreatment reversed EPI+MALP2 effects on BM-MSC migration (EPI+MALP2: 21% inhibition vs. T+Epi+MALP2: 8.7% inhibition; P<0.001) (FIG. 7a). Timolol's blocking effects were prominent in NHK with the cells returning to the migratory speeds of untreated cells (T+EPI+MALP2: 16% vs Epi+MALP2: 60% inhibition and T+EPI+HKSA: 18% vs EPI+HKSA: 52%; P<0.0003) (FIGS. 7b & 7c).

The antagonist effect of Timolol on IL-6 release was observed in both BM-MSC and NHK. Levels of IL-6 were three fold higher in HKSA treated BM-MSC and NHK compared to MALP2 treatment (FIG. 7d-g). Pretreatment with Timolol decreased EPI+MALP2 or EPI+HKSA induced IL-6 secretion by 33-fold and 1.5 fold, respectively (FIG. 7d, 7f). Similar inhibitory effects with Timolol pretreatment, though lower in magnitude, were observed in NHK (FIG. 7e, 7g). Blocking the β2-AR using the receptor specific inhibitor, ICI (53,54) also significantly reduced EPI+MALP2 (21.8±3 vs ICI+EPI+MALP2: 4±0.3 pg/μg protein, P<0.05) or EPI+HKSA (67.7±3 vs ICI+EPI+HKSA: 5.1±0.8 pg/μg protein, P<0.05) induced IL-6 levels in BM-MSC, suggesting that the observed response can be ascribed to β2-AR. Additionally, we examined the levels of IL-6 in Timolol alone, Timolol+MALP2-treated cells. Timolol alone induced marginally higher IL-6 levels (Tim: 4.8±2.7 pg/μg protein) compared to untreated cells (Control: 1.8±0.6 pg/μg protein) and did not affect MALP2-induced IL-6 secretion in BM-MSC (Timolol+MALP2: 8.08±0.5 pg/μg protein), and perhaps because of its known ability to act as an inverse agonist (55). However, ICI was able to decrease MALP2 induced IL-6 secretion in cells (EPI+MALP2: 21.8±3 vs ICI+EPI+MALP2: 6.4±1 pg/μg protein, P<0.05). Taken together, these data suggest that the effects are specific to β2-AR and Timolol or ICI greatly diminishes the inflammatory response and significantly improved cell migration.

### 6. β2-AR and TLR2 Form Physical Association for Cross Signaling

Using co-immunoprecipitation assays with β2-AR and TLR2 antibody, we demonstrated the association between β2-AR-TLR2-MyD88-IRAK-1 and TLR2-β2-AR-BARK-1 signaling in MALP2 and EPI treated NHKs (FIGS. 7h and 7i). There is a noticeable increase in β2-AR expression and BARK-1 phosphorylation in MALP2 treated cells and a similar increase in TLR2, MyD88 recruitment, IRAK-1 phosphorylation in EPI cells (FIGS. 7h and 7i), suggesting a physical association between the β2-AR and TLR2 by the formation of a signaling platform within the cell membrane. Protein A/G-Sepharose beads and isotype matched IgG antibodies did not show a strong association.

### 7. TLR2 Ligands (MALP2/HKSA) Induce Catecholamine Secretion in BM-MSC and NHK

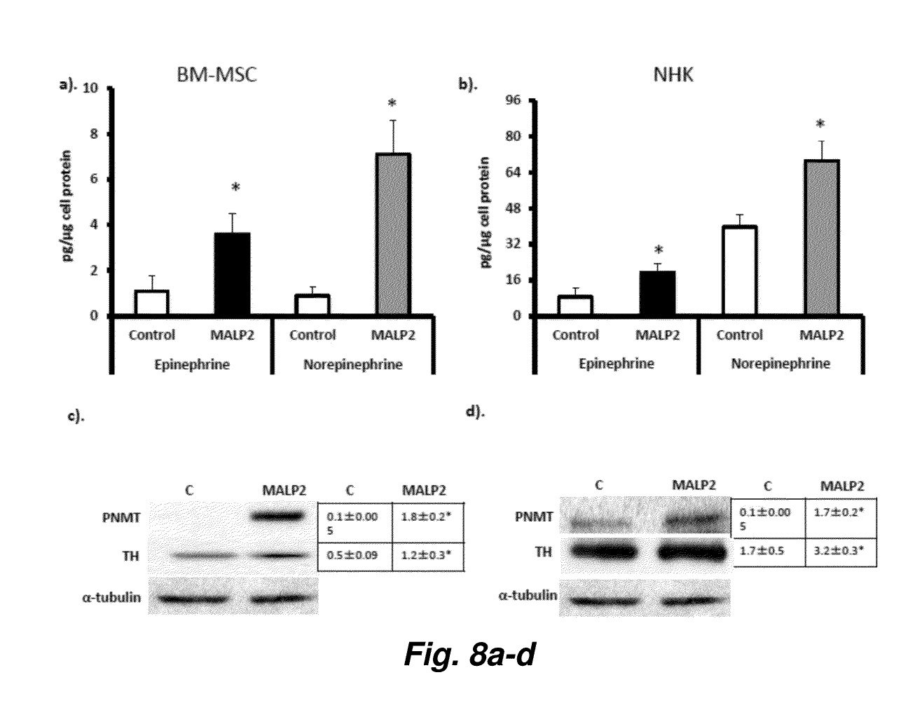

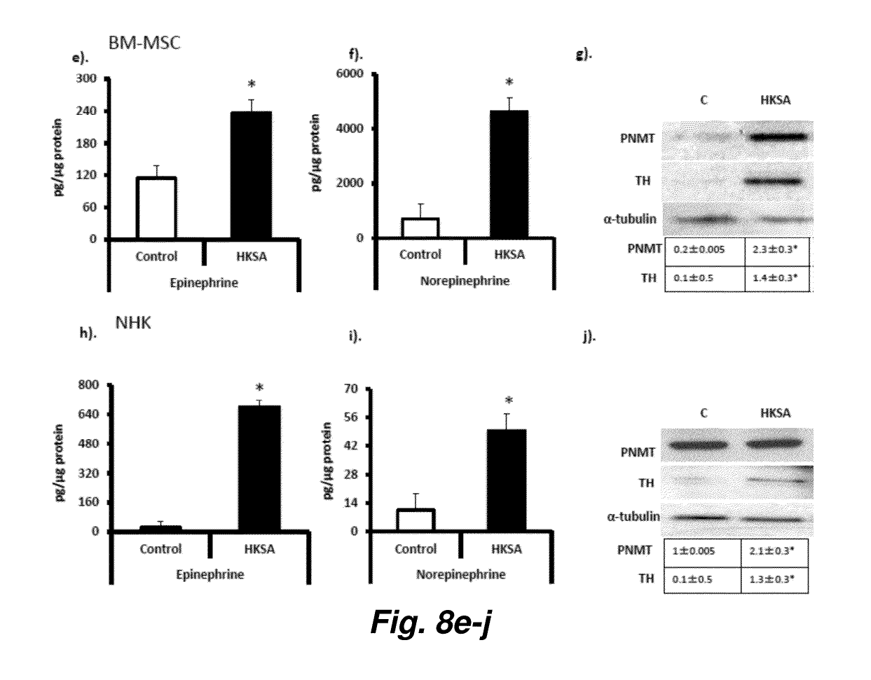

The work presented above demonstrates that activation of the adrenergic receptor in the presence of TLR ligands can upregulate the TLR-mediated immune response, and conversely, that activation of TLR by its bacterially-generated ligands cross-talks to activate signaling through the adrenergic receptor pathway. Here we ask the related question of whether the bacterial (TLR2) ligands can further contribute to adrenergic receptor signaling response by increasing secretion of their catecholamine ligands by wound-resident cells. We found that EPI and norepinephrine levels in the cell culture supernatants of MALP2 treated BM-MSC and NHK (FIGS. 8a and 8b) were increased by TLR2 ligation, with the NHKs response is 2-4 fold higher. On the basis of these findings, we examined the presence of two key enzymes involved in catecholamine synthesis, namely PNMT and TH (two rate-limiting enzymes). MALP2 induced PNMT and TH in BM-MSC and NHK (FIGS. 8c, 8d). Exposure of BM-MSC and NHK to HKSA produced similar upregulations in these enzymes (FIGS. 8e-8j). These results provide the first evidence that catecholamines can be generated by BM-MSC and that their levels can be modulated by TLR2 activation, as do NHK. Thus the wound presents both paracrine and autocrine-signaling pathways for locally generated catecholamines, which then in turn, upregulate pro-inflammatory responses in BM-MSC and NHK via TLR2 activation and impact cell migration. Furthermore, this suggests that modulation of the generation of catecholamines by β2-AR inhibitors could potentially decrease the intensity of inflammation in wounds.

### 8. EPI and TLR2 Ligands Decrease In Vitro Scratch Wound Closure

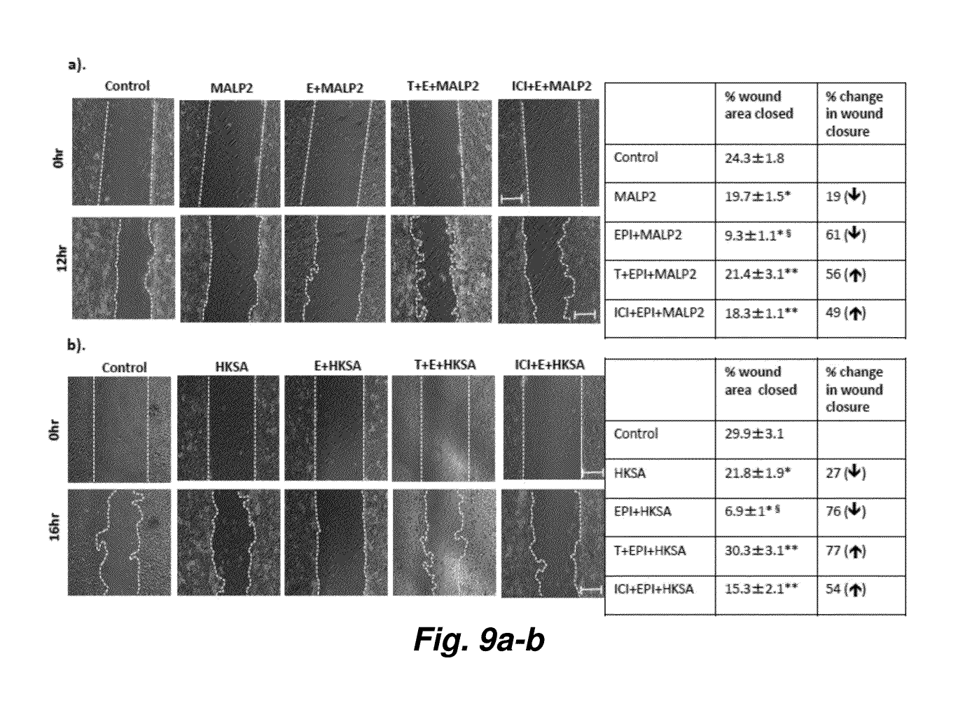

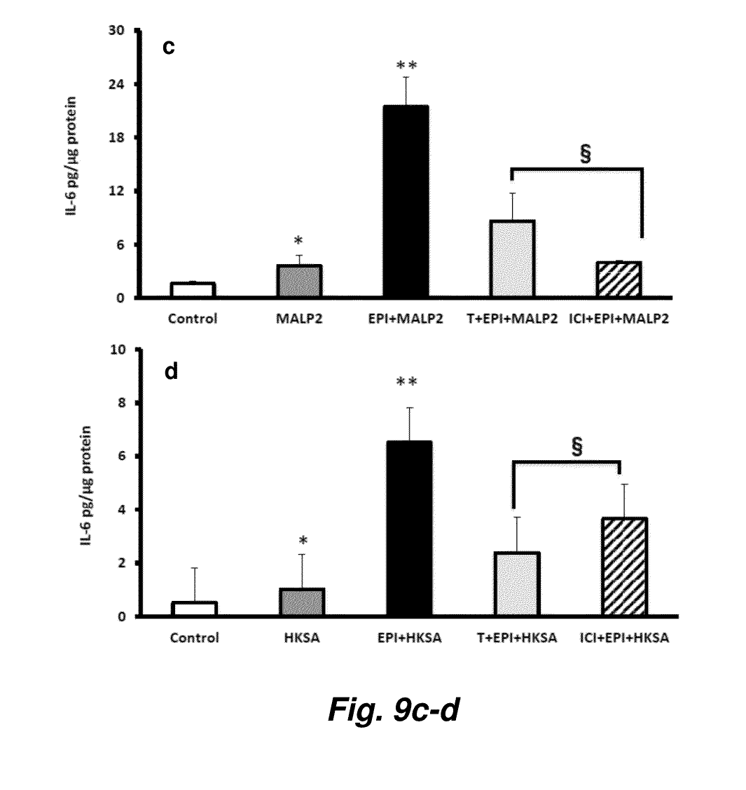

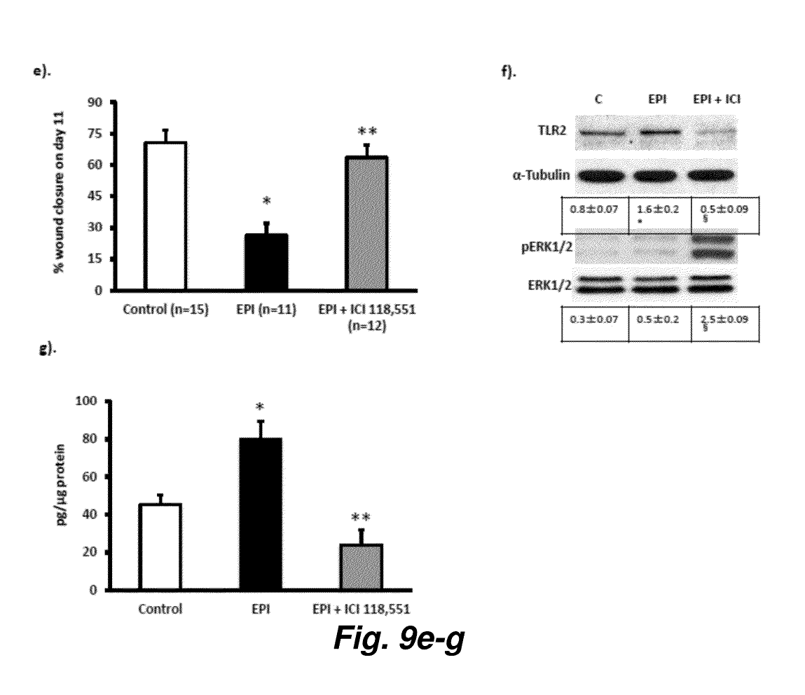

Since keratinocyte migration from the wound edge is critical for healing, we used a scratch wound assays to determine if EPI and MALP2 can modulate NHK mobility in a wound environment (30, 56). EPI and MALP2 decrease NHK scratch wound closure (Control: 37% closed, EPI+MALP2: 9.3% closed, P<0.05) while the addition of antagonists reverses this effect (T+EPI+MALP2: 23% closed; FIG. 9a). Similar patterns were observed with HKSA (EPI+HKSA: 7% closed after 16 hrs; FIG. 9b). Furthermore, we observed a corresponding increase in IL-6 secretion in wounded NHK confluent sheets treated with EPI and MALP2/HKSA (FIGS. 9c & 9d).

### 9. Cutaneous Wounds in EPI Stressed Mice Show Decreased Wound Closure, Increased TLR2 Expression, Decreased Phosphorylation of ERK1/2, and Increased IL-6 Expression.

To determine if the observed in vitro effects of EPI-induced TLR2 expression, signaling, and wound healing in BM-MSC and NHK translated to the in vivo situation, we used a pharmacologic model of sustained EPI stress to impair healing in mice (13,30). Wound closure was significantly decreased in EPI stressed mice compared to control mice and was reversed in animals treated with the β2-AR antagonist ICI (FIG. 9e). Wound tissues excised from the EPI-stressed animals (day 7) demonstrate increased TLR2 expression compared to untreated mice seven days after injury, indicative of prolongation of inflammation, and this is reversed in the presence of ICI (FIG. 9f). Decreased phosphorylation of ERK1/2 is also observed in the EPI-stressed wounds, which may contribute to the delay in healing, as others and we have shown that ERK1/2 phosphorylation is required for epithelial wound healing in vitro and in vivo (12, 30, 57-59). IL-6 levels in the wound tissue of the EPI-stressed animals were likewise significantly elevated, and decreased in the presence of ICI (FIG. 9g). These data suggest that the impairment of healing observed with sustained elevation of EPI levels may be contributed by the EPI-induced upregulation of TLR2 expression, and IL-6 levels within the wound beds.

### Discussion

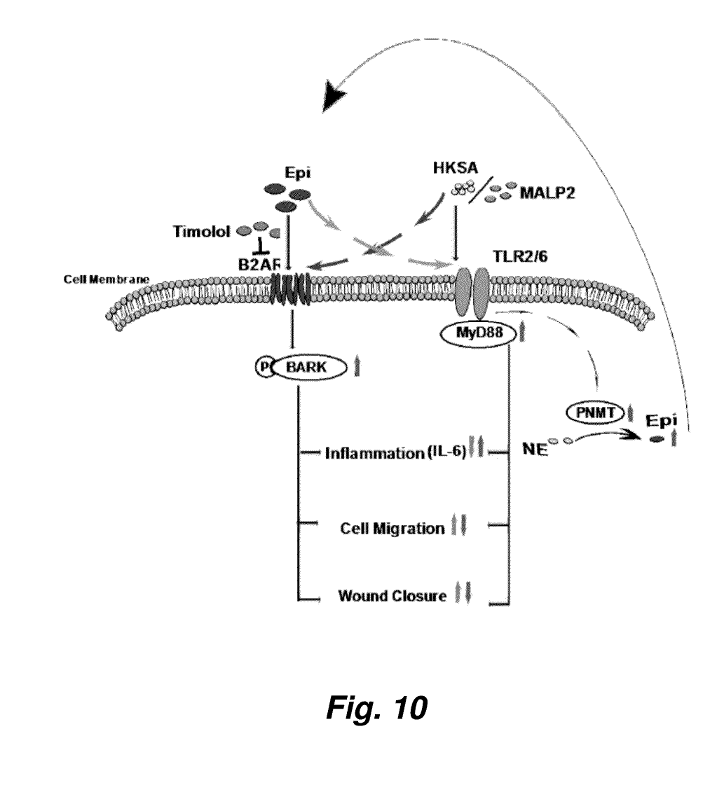

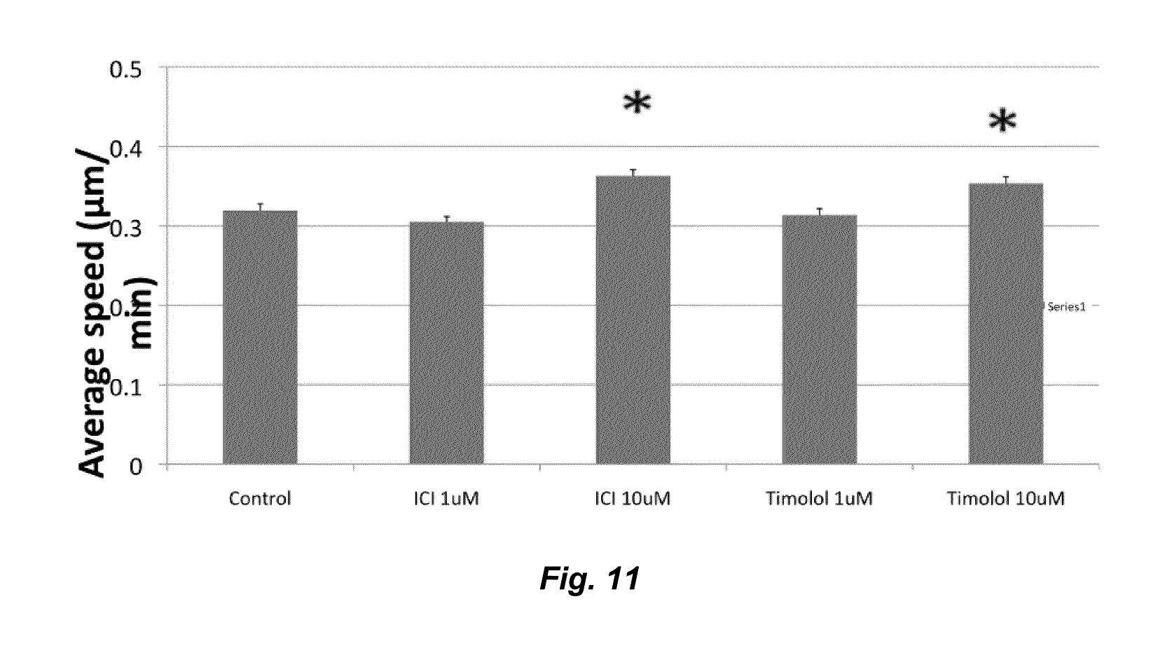

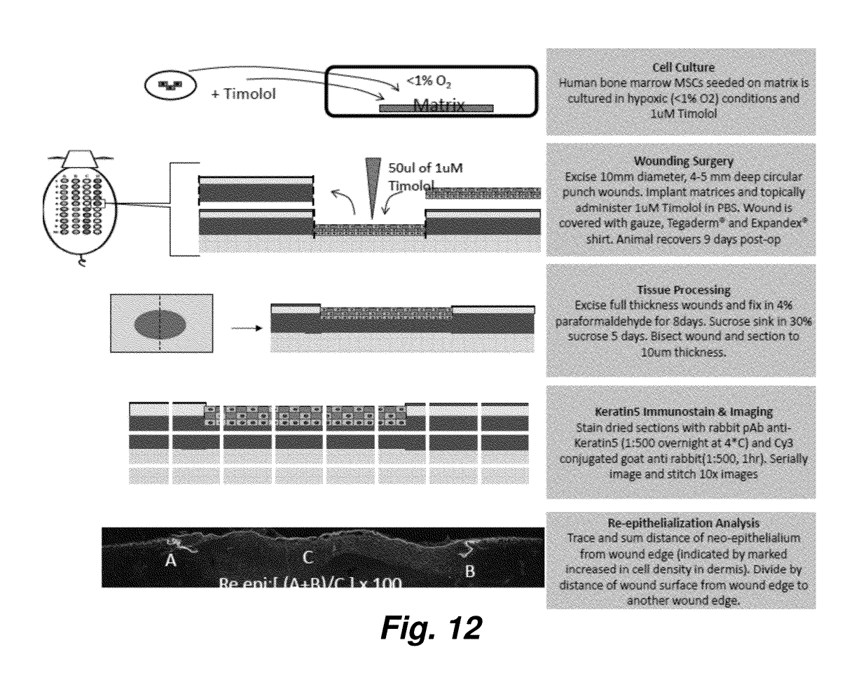

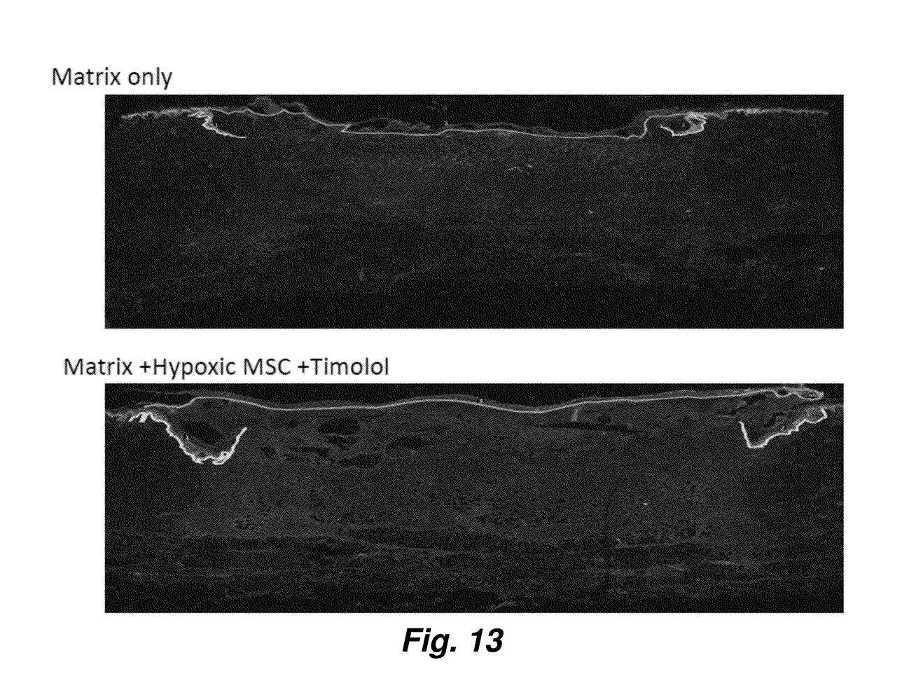

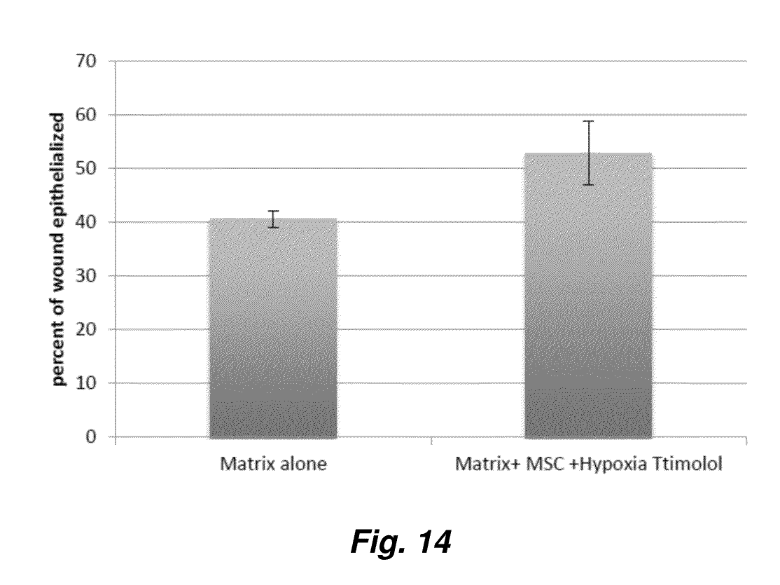

The ability of catecholamine stress to impair healing has been well documented with mechanisms of impairment ascribed to alteration in keratinocyte and fibroblast function (11-13) as well as prolongation of neutrophil persistence in the wound (60). Here we present data to demonstrate a novel mechanism by which EPI stress synergizes with innate immune receptor (TLR2) function in BM-MSC and keratinocytes to generate a pro-inflammatory environment that can ultimately impair wound healing. Cross-talk between the adrenergic receptors (AR) and the TLRs present on these cell types result in their impaired ability to migrate, as well as upregulated generation of the pro-inflammatory cytokine IL-6. Interestingly, we find that EPI activation of the AR can induce increased TLR2 receptor expression, activation and downstream signaling, and conversely, TLR2 activation, either by the agonist MALP2 or by bacteria, can induce increased β2-AR receptor expression, activation and downstream signaling. Activation of either the β2-AR or the TLR2 receptor results in the physical association of the two receptors and their proximal downstream effectors, mechanistically providing a signaling platform for the cross-talk. Concurrent activation of the AR and TLR pathways results in synergistic effects on cell migration and inflammation. These deleterious effects are amplified by an autocrine loop, wherein TLR activation results in increased synthesis of EPI by upregulation of catecholamine synthetic enzymes in both keratinocytes and BM-MSC. Thus, the present work describes a new paradigm for functional interplay between stress hormones and bacterial ligands wherein the dual ligand signaling results in cross activation of both the adrenergic receptor system and the innate immune/inflammatory pathways in MSC, with resultant potential deleterious consequences for healing (FIG. 10). Since both bacteria and elevated levels of EPI are hallmarks of chronic wounds, this cross-talk pathway presents a “recipe for impaired wound healing.”

Systemic levels of the catecholamine stress hormones, EPI and norepinephrine, are several fold elevated above physiologic levels during anxiety, pathogenic challenge, or injury and trauma (8-10). These ligands are agonists for the adrenergic receptors (α and β-AR) that are expressed on all immune competent cells (61) including those involved in innate immune responses (62-64). While much of the earlier literature supported the notion that activation of the β2-AR suppresses immune response and inflammation (65), emerging literature has shown that AR activation can result in pro-inflammatory responses from the immune system. For example, activation of β2-AR has been shown to be responsible for inflammatory immune cell responses characterized by increased cytokine (IL-1β, IL-6, TNF-α) production (66, 67). Furthermore, activation of the β2-AR with salmeterol in the RAW 264.7 macrophages resulted in 80- and 8-fold increase in IL-1β and IL-6 transcripts, respectively, accompanied by a significant increase in IL-1β and IL-6 protein production (40). Local elevation of IL-6 levels in the wound, mediated by catecholamine activation of the β2-AR in wound macrophages, results in increased dwell time of neutrophil trafficking to the wound, thus delaying healing (60). Thus, AR activation on immune cells is associated with variable local pro-inflammatory factor release and may affect the wound healing process.

MSCs are another cell type with potent immuno-modulatory capacity. These cells are recruited to a wound or site of injury (20,21) and can attract immune inflammatory cells (68,69). Interestingly, murine MSCs express a full repertoire of AR, including β1, β2, β3 (70-72). Their activation has been previously investigated primarily in the realm of MSC lineage commitment (70-72) and to some extent for the ability to impact upon their immune orchestrating abilities. Since both keratinocytes and MSC express AR, and both are critical for wound repair, we chose to investigate how activation of these receptors by their stress-induced catecholamine ligands could impact on functions critical for healing, such as migration and inflammation.

Like ARs, TLRs play a crucial role in the wound biology and innate immunity. TLRs activation constitutes one of the earliest responses of an organism to microbial invasion (73,74). We, and others, have demonstrated that prolonged stimulation of TLRs leads to increased inflammation with a corresponding decrease in the ability to heal (31, 75). Of note, an increasing body of evidence indicates that catecholamines can modulate innate cytokine responses with increased expression of pro-inflammatory cytokines (66, 76). For instance, EPI can upregulate LPS-stimulated human monocytic cytokine responses (via TLR4, IL-12, TNF-α-, and IL-10) (66). In murine macrophages, epinephrine pretreatment significantly increases TNF-α production with LPS stimulation and this effect is mitigated by blockade of either the α2-AR or β2-AR (10). These effects can be very cell type and species specific since the reverse finding, of a β2-AR agonist mediated decrease in TLR innate immune responses, has also been reported (77). In a monocytic cell line and in rat macrophages, exposure to supraphysiologic levels of EPI (5000-10,000 ng/ml) decreases TLR4 mRNA expression (78, 79). All of these studies focused on TLR4 responses, mostly mediated by gram-negative bacterial ligands (LPS). However, the major isolates within chronic wounds are gram-positive bacteria (ex. Staphylococcus aureus, 46-48) and thus to maintain physiological relevance, we examined the effects of EPI on TLR2 (activated by MALP2 or S. aureus; 35-37, 45) in human MSC and keratinocytes.

Given the important physiological roles for both TLR2 and β2-ARs in wound biology and the regulatory role of BARK-1 in β2-AR downstream signaling, we examined the hypothesis that TLR2 activation modulates BARK-1 phosphorylation. We found that EPI increased BARK-1 phosphorylation in BM-MSC, as might be expected. More surprisingly, however, is the finding that activation of TLR2 with MALP2 also increased BARK-lphosphorylation both in BM-MSC and NHK. Although earlier studies in murine peritoneal macrophages (39) have suggested that the mechanisms for TLR ligand induced BARK-1 expression is regulation at both transcriptional and post-transcriptional levels (39) and our findings demonstrate that there may be a physical association between β2-AR and TLR2 receptors and the possibility of a cross activation by the respective ligands at the receptor and signaling levels. Furthermore, our results may explain findings in other inflammatory disease processes. For example, an increase in BARK-1 expression was observed in neutrophils obtained from septic humans relative to those of healthy individuals (79), sepsis being a condition associated with both activation of TLR and AR (80,81) by high systemic levels of stress catecholamines and bacterial derived ligands (8-10, 34, 80, 81). These results support our hypothesis that a combination of catecholamine stress and signaling through the innate immune intracellular transduction pathway result in an exaggerated inflamed local environment, characteristic of chronic wounds.

In 1994, Bergquist et al (82) demonstrated the presence of endogenous catecholamines in lymphocytes and provided evidence for an autocrine regulation of catecholamine synthesis in non-neuronal cells. A decade and half later, Flierl and colleagues showed that phagocyte-derived catecholamines enhance injury (61, 83). The authors demonstrated that exposure of phagocytes to LPS led to an increase in catecholamine release with corresponding changes in the catecholamine enzymatic generating machinery and suggested that regulation of catecholamine generation and degradation may alter the release of proinflammatory mediators in cells (62,84). In line with these two pioneering studies, we now show that both BM-MSC and NHK may serve as new sources for non-neuronal catecholamine generation within a wound environment. The autocrine loop generated by TLR activation on either BM-MSC or keratinocytes has the potential to locally generate EPI that then can amplify the inflammatory response by promoting release of IL-6.

Here we show how EPI and TLR2 ligands potentiate proinflammatory IL-6 production via the β2-AR/BARK-1 or TLR2-MyD88 signaling pathway, and that β2-AR antagonists reverse the inflammatory cytokine production and the migration defects in cells exposed to both receptors' ligands. Isolated cells in culture often respond differently than do those same cell types within a complex tissue environment. To determine if our observations translate to the in vivo environment, we examined wounds in EPI-stressed mice. Healing was impaired in the EPI-stressed animals, and the impairment reversed by blockade of the β2-AR. Wound tissues of the EPI-stressed animals demonstrate increased TLR2 expression, as well as increased IL-6 levels relative to unstressed animals. These finding provide a framework for the development of therapeutic strategies that could selectively regulate inflammatory responses in the impaired healing wound.

Indeed, several studies have already reported that treatment with β2-AR blockers improves outcomes, such as decreased pro-inflammatory cytokine secretion and improved immune cell function, in patients who have endured an operative or traumatic injury (10, 84-88). Improved healing in chronic skin wounds has also been reported using topical application of β-AR antagonists (51, 89). The mechanisms underlying the noted improved outcomes have only been partially explained. However, our study provides additional mechanistic insights by using pharmacological and biochemical approaches to characterize the signal-transduction properties of the synergistic relationship between β2-AR and TLR2 activation that results in an amplified IL-6 response. The synergistic IL-6 effect shown in our study depends on β2-AR stimulation as evidenced by a reversal of this response by either of two different β2-AR antagonists (Timolol and ICI; FIG. 9). TLR2 effector pathways are linked to the myeloid differentiation factor-88 (MyD88) signaling complex, which activates the nuclear factor κ-light-chain-enhancer of activated B cells (NF-κB) to regulate IL-6 transcription (14). Our data show that EPI induces MyD88 expression and suggests that β2-AR interaction with TLR2 may also involve MyD88 recruitment. The limitations of the current study include the lack of data on genomic ablations of either β2-AR or TLR2 genes and examining the corresponding ligand induced effects in the cells. Our continuing studies are focused on fully understanding the intricate interactions between the two signaling pathways using a combination of pharmacological, biochemical, and or genomic approaches.

The presence of bacteria in chronic wounds can influence the balance between successful and adverse healing outcomes. Staphylococcus aureus is noted to be the pathogen harbored by the great majority of chronic wounds (45-48, 90). In addition to bacterial presence in wounds, many wounds are in a high catecholamine environment. In particular, patients with burn wounds and chronic inflammatory diseases have elevated levels of catecholamines (8-10, 13). Although both catecholamine stress and TLR2 activation individually contribute to the chronic wound pathology there are no studies linking the two. Our study makes this connection with wide ranging clinical implications for persistent inflammation, stress, and infection.

In conclusion, we have shown that EPI-mediated activation of the innate immune receptor TLR2, IL-6 production, and impaired wound healing might represent a previously unrecognized hormonal, immunological mechanism that is involved in shaping the roles of BM-MSC and NHK in the wound healing process. This neuroendocrine mechanism may play a critical role in driving innate immune receptor profiles in wounds with intrinsic overexpressed catecholamines. Thus, in the infected wounds, migrating and resident cells react to bacterial ligands/infection by inducing catecholamine production and potentiate persistent inflammation creating an impaired healing phenotype. Our findings have implications for the hormonal innate immune receptor interactions and for understanding the mechanisms controlling the differing susceptibility to infections and immune/inflammatory-related conditions in wounds.

### REFERENCES

1. Sen C K, Gordillo G M, Roy S, et al. Human skin wounds: a major and snowballing threat to public health and the economy. WOUND REPAIR REGEN 2009; 17(6): 763-71.
2. Falanga V. Wound healing and its impairment in the diabetic foot. LANCET 2005; 366(9498): 1736-43.
3. Brem H, Sheehan P, Boulton A J. Protocol for treatment of diabetic foot ulcers. A M J SURG 2004; 187 (5A):1S-10S.
4. Davis S C, Ricotti C, Cazzaniga A, Welsh E, Eaglstein W H, Mertz P M. Microscopic and physiologic evidence for biofilm-associated wound colonization in vivo. WOUND REPAIR REGEN 2008; 16(1):23-9.
5. Dasu M R, Isseroff R R. Toll-like receptors in wound healing: location, accessibility, and timing. J INVEST DERMATOL 2012; 132(8): 1955-8.
6. Dixon R A, Kobilka B K, Strader D J, et al. Cloning of the gene and cDNA for mammalian beta-adrenergic receptor and homology with rhodopsin. NATURE 1986; 321(6065): 75-9.
7. Rockman H A, Koch W J, Lefkowitz R J. Seven-transmembrane-spanning receptors and heart function. NATURE 2002; 415(6868): 206-12.
8. Moore F A, Moore E E. Evolving concepts in the pathogenesis of post injury multiple organ failure. SURG CLIN NORTH AMERICA 1995; 75(2): 257-77.
9. Woolf P D, McDonald J V, Feliciano D V, et al. The catecholamine response to multisystem trauma. ARCH SURG 1992; 127(8): 899-903.
10. Rough J, Engdahl R, Opperman K, et al. Beta-2-adrenoreceptor blockade attenuates the hyperinflammatory response induced by traumatic injury. SURGERY 2009; 145(2): 235-42.
11. Chen J, Hoffman B B, Isseroff R R. Beta-adrenergic receptor activation inhibits keratinocyte migration via a cyclic adenosine monophosphate-independent mechanism. J INVEST DERMATOL 2002; 119: 1261-1268.
12. Pullar C E, Grahn J C, Liu W, Isseroff R R. Beta2-adrenergic receptor activation delays wound healing. FASEB J 2006; 20: 76-86.
13. Sivamani R K, Pullar C E, Manabat-Hidalgo C G, et al. Stress-mediated increases in systemic and local epinephrine impair skin wound healing: potential new indication for beta blockers. PLOS MED 2009; 6 (1):e12.
14. Akira S, Takeda K. Toll-like receptor signalling. NAT REV IMMUNOL 2004; 4(7): 499-511.
15. Dasu M R, Ramirez S, Isseroff R R. Toll-like receptors and diabetes: a therapeutic perspective. CLIN SCI (Lond). 2012; 122(5): 203-14.
16. Schauber J, Dorschner, R A, Coda, A B, et al. Injury enhances TLR2 function and antimicrobial peptide expression through a vitamin D-dependent mechanism. J CLIN INVESTIG. 2007; 117(3):803-11.
17. Oppong G O, Rapsinski G J, Newman T N, et al. Epithelial cells augment barrier function via activation of the Toll-like receptor 2/phosphatidylinositol 3-kinase pathway upon recognition of Salmonella enterica serovar Typhimurium curli fibrils in the gut. INFECT IMMUN. 2013; 81(2):478-86.
18. Badiavas A R, Badiavas E V. Potential benefits of allogeneic bone marrow mesenchymal stem cells for wound healing. EXPERT OPIN BIOL THERAPY 2011; 11(11): 1447-54.
19. Falanga V, Iwamoto S, Chartier M, et al. Autologous bone marrow-derived cultured mesenchymal stem cells delivered in a fibrin spray accelerate healing in murine and human cutaneous wounds. TISSUE ENG 2007; 13(6): 1299-312.
20. Wu Y, Chen L, Scott P G, et al. Mesenchymal stem cells enhance wound healing through differentiation and angiogenesis. STEM CELLS 2007; 25(10): 2648-59.
21. Jackson W M, Nesti L J, Tuan R S. Concise review: clinical translation of wound healing therapies based on mesenchymal stem cells. STEM CELLS TRANS MED. 2012; 1(1):44-50.
22. Singer A J, Clark R A. Cutaneous Wound healing. N ENG J MED 1999; 341:738-746.
23. Chang W, Lim S, Song B W, et al. Phorbol myristate acetate differentiates human adipose-derived mesenchymal stem cells into functional cardiogenic cells. BIOCHEM BIOPHYS RES COMMUN. 2012; 424(4):740-6.
24. Raicevic G, Najar M, Stamatopoulos B, et al. The source of human mesenchymal stromal cells influences their TLR profile as well as their functional properties. CELL IMMUNOL 2011; 270(2): 207-16.
25. Lebre M C, van der Aar A M, van Baarsen L, et al. Human keratinocytes express functional Toll-like receptor 3, 4, 5, and 9. J INVEST DERMATOL 2007; 127(2):331-41.
26. Grisanti L A, Woster A P, Dahlman J, et al. alphal-adrenergic receptors positively regulate Toll-like receptor cytokine production from human monocytes and macrophages. J PHARMACOL EXP THERAP 2011; 338(2): 648-57.
27. Olson S D, Pollock K, Kambal A, et al. Genetically engineered mesenchymal stem cells as a proposed therapeutic for Huntington's disease. MOL NEUROBIOL 2012; 45(1):87-98.
28. Rheinwald J G, Green H. Serial cultivation of strains of human epidermal keratinocytes: the formation of keratinizing colonies from single cells. CELL 1975; 6: 331-343.
29. Isseroff R R, Ziboh V A, Chapkin R S, Martinez D T. Conversion of linoleic acid into arachidonic acid by cultured murine and human keratinocytes. J LIPID RES 1987; 28(11): 1342-9.
30. Pullar C E, Rizzo A, Isseroff R R. beta-adrenergic receptor antagonists accelerate skin wound healing: evidence for a catecholamine synthesis network in the epidermis. J BIOL CHEM 2006; 281: 21225-21235.
31. Dasu M R, Thangappan R K, Bourgette A, et al. TLR2 expression and signaling-dependent inflammation impair wound healing in diabetic mice. LAB INVEST 2010; 90(11): 1628-36.
32. Steenhuis P, Huntley R E, Gurenko Z, et al. Adrenergic signaling in human oral keratinocytes and wound repair. J DENTAL RES 2011; 90(2):186-92. 33. Leis S, Drenkhahn S, Schick C, et al. Catecholamine release in human skin- a microdialysis study. EXPERIMENTAL NEUROBIOLOGY 2004; 188; 86-93.
34. Little R A, Frayn K N, Randall P E, et al. Plasma catecholamine concentrations in acute states of stress and trauma. ARCH EMERGENCY MEDICINE 1985; 2(1): 46-7.
35. Mulhlradt P F, Kiess M, Meyer H, et al. Isolation, structure elucidation, and synthesis of a macrophage stimulatory lipopeptide from Mycoplasma fermentans acting at picomolar concentration. J EXP MED 1997; 185(11):1951-8.
36. Galanos C, Gumenscheimer M, Mu\ddot{}hlradt P F, et al. MALP-2, a Mycoplasma lipopeptide with classical endotoxic properties: end of an era of LPS monopoly? J ENDOTOXIN RES 2000; 6, 471-476.
37. Into T, Nodasaka Y, Hasebe A, et al. Mycoplasmal lipoproteins induce toll-like receptor 2- and caspases-mediated cell death in lymphocytes and monocytes. MICROBIOL IMMUNOL 2002; 46(4):265-76.
38. Tsuzuki H, Tani T, Ueyama H, Kodama M. Lipopolysaccharide: neutralization by polymyxin B shuts down the signaling pathway of nuclear factor kappaB in peripheral blood mononuclear cells, even during activation. J SURG RES 2001; 100(1): 127-34.
39. Wang Y, De Arcangelis V, Gao X, et al. Norepinephrine- and epinephrine-induced distinct beta2-adrenoceptor signaling is dictated by GRK2 phosphorylation in cardiomyocytes. J BIOL CHEM 2008; 283(4): 1799-807.
40. Tan K S, Nackley A G, Satterfield K, et al. Beta2 adrenergic receptor activation stimulates pro-inflammatory cytokine production in macrophages via PKA- and NF-kappaB-independent mechanisms. CELL SIGNAL. 2007; 19(2):251-60.
41. Szabo-Fresnais N, Lefebvre F, Germain A, et al. A new regulation of IL-6 production in adult cardiomyocytes by beta-adrenergic and IL-1 beta receptors and induction of cellular hypertrophy by IL-6 trans-signalling. CELL SIGNAL 2010; 22(7):1143-52.
42. Wang Y, De Arcangelis V, Gao X, et al. Norepinephrine- and epinephrine-induced distinct beta2-adrenoceptor signaling is dictated by GRK2 phosphorylation in cardiomyocytes. J BIOL CHEM 2008; 283(4): 1799-807.
43. Nguyen C M, Nolta J A, Isseroff R R. Pro-migratory effects of stromal cell-derived factor 1 on human mesenchymal stem cells under stress-induced environment. J INVEST DERMATOL 2012; 132: S135-S148.
44. Lin B R, Bolaji R S, Nolta J A, et al. Stress related catecholamines alter chemokinetics of hMSC in vitro. J INVEST DERMATOL 2009; 129:S8-S8.
45. Takeuchi, O., K. Hoshino, S. Akira Cutting edge: TLR2-deficient and MyD88-deficient mice are highly susceptible to Staphylococcus aureus infection. J IMMUNOL 2000; 165: 5392-5396
46. Colsky A S, Kirsner R S, Kerdel F A. Microbiologic evaluation of cutaneous wounds in hospitalized dermatology patients. OSTOMY WOUND MANAGE 1998; 44: 40-46.
47. Gjodsbol K, Christensen J J, Karlsmark T, et al. Multiple bacterial species reside in chronic wounds: a longitudinal study. INT WOUND J 2006; 3:225-231.
48. Valencia I C, Kirsner R S, Kerdel F A. Microbiologic evaluation of skin wounds: alarming trend toward antibiotic resistance in an inpatient dermatology service during a 10-year period. J AM ACAD DERMATOL 2004; 50:845-849.
49. Pereira C T, Jeschke M G, Herndon D N. Beta-blockade in burns. NOVARTIS FOUND SYMP 2007; 280:238-48; discussion 248-51.
50. Zhang X J, Meng C, Chinkes D L, et al. Acute propranolol infusion stimulates protein synthesis in rabbit skin wound. SURGERY 2009 May; 145(5):558-67.
51. Tang J C, Dosal J, Kirsner R S. Topical timolol for a refractory wound. DERMATOL SURG 2012; 38(1):135-138.
52. Shelling M L, Federman D G, Kirsner R S. Clinical approach to atypical wounds with a new model for understanding hypertensive ulcers. ARCH DERMATOL 2010; 146(9): 1026-1029.
53. Denda M, Fuziwara S, Inoue K. Beta2-adrenergic receptor antagonist accelerates skin barrier recovery and reduces epidermal hyperplasia induced by barrier disruption. J INVEST DERMATOL 2003; 121(1):142-8.
54. Pullar C E, Isseroff R R. Beta 2-adrenergic receptor activation delays dermal fibroblast-mediated contraction of collagen gels via a cAMP-dependent mechanism. WOUND REPAIR REGEN 2005; 13(4):405-11.
55. West G M, Chien E Y, Katritch V, et al. Ligand-dependent perturbation of the conformational ensemble for the GPCR β2-adrenergic receptor revealed by HDX. STRUCTURE 2011; 19(10): 1424-32.
56. Peplow P V, Chatterjee M P. A review of the influence of growth factors and cytokines in in vitro human keratinocyte migration. CYTOKINE 2013; 62(1):1-21.
57. Zhang M, Sun L, Wang X, et al. Activin B promotes BM-MSC-mediated cutaneous wound healing by regulating cell migration via the JNK-ERK signaling pathway. CELL TRANSPLANT. 2013 Apr. 12. [Epub ahead of print]
58. Shibata S, Tada Y, Asano Y, et al. (2012) Adiponectin regulates cutaneous wound healing by promoting keratinocyte proliferation and migration via the ERK signaling pathway. J IMMUNOL. 2012; 189(6):3231-41.
59. Lima M H, Caricilli A M, de Abreu L L, et al. Topical insulin accelerates wound healing in diabetes by enhancing the AKT and ERK pathways: a double-blind placebo-controlled clinical trial. PLoS ONE 2012; 7 (5):e36974.
60. Kim M, Gorouhi F, Ramirez S, et al. Systemic epinephrine mediates an impairment of wound healing by altering tissue neutrophil trafficking via β2 adrenergic receptor and IL-6 dependent mechanism. J INVEST DERMATOL 2013 Oct. 11. doi: 10.1038/jid.2013.415. [Epub ahead of print] PMID: 24121404.
61. Sanders V M, Kasprowicz D J, Kohm A P, et al. In: Neurotransmitter receptors on lymphocytes and other lymphoid cells. Ader R, Felten D, Cohen N, eds. Psychoneuroimmunology Academic Press 2001; 161-196.
62. Flierl M A, Rittirsch D, Huber-Lang M, et al. Catecholamines-crafty weapons in the inflammatory arsenal of immune/inflammatory cells or opening pandora's box? MOL MED 2008; 14 (3-4):195-204.
63. Johnson M. The beta-adrenoceptor. AM J RESPIR CRIT CARE MED 1998; 158 (5 Pt3): 5146-53.
64. Maestroni G J. Dendritic cell migration controlled by alpha 1b-adrenergic receptors. J IMMUNOL 2000; 165(12): 6743-7.
65. Farmer P, Pugin J. beta-adrenergic agonists exert their “anti-inflammatory” effects in monocytic cells through the IkappaB/NF-kappaB pathway. AM J PHSIOL LUNG CELL MOL PHYSIOL 2000; 279 (4):L675-82.
66. Elenkov I J, Kvetnansky R, Hashiramoto A, et al. Low-versus high-baseline epinephrine output shapes opposite innate cytokine profiles: presence of Lewis- and
Fischer-like neurohormonal immune phenotypes in humans? J IMMUNOL 2008; 181(3):1737-45.
67. Grisanti L A, Evanson J, Marchus E, et al. Pro-inflammatory responses in human monocytes are beta1-adrenergic receptor subtype dependent. MOL IMMUNOL 2010; 47(6): 1244-54.
68. Tomchuck S L, Zwezdaryk K J, Coffelt S B, et al. Toll-like receptors on human mesenchymal stem cells drive their migration and immunomodulating responses. STEM CELLS 2008; 26(1): 99-107.
69. Romieu-Mourez R, Fran Bois M, Boivin M N, et al. Cytokine modulation of TLR expression and activation in mesenchymal stromal cells leads to a proinflammatory phenotype. J IMMUNOL 2009; 182(12): 7963-73.
70. Li H, Fong C, Chen Y, et al. Beta-adrenergic signals regulate adipogenesis of mouse mesenchymal stem cells via cAMP/PKA pathway. MOL CELL ENDOCRINOL 2010; 323(2):201-7.
71. Takahata Y, Takarada T, Iemata M, et al. Functional expression of beta2 adrenergic receptors responsible for protection against oxidative stress through promotion of glutathione synthesis after Nrf2 upregulation in undifferentiated mesenchymal C3H10T1/2 stem cells. J CELL PHYSIOL 2009; 218(2):268-75.
72. Han J, Zou Z, Zhu C, et al. DNA synthesis of rat bone marrow mesenchymal stem cells through alphal-adrenergic receptors. ARCH BIOCHEM BIOPHYS 2009; 490(2):96-102.
73. Ulevitch R J, Mathison J C, da Silva Correia J. Innate immune responses during infection. VACCINE 2004; 22 (1):S25-30.
74. Kawai T, Akira S. SnapShot: Pattern-recognition receptors. CELL 2007; 129(5):1024.
75. Mollen K P, Anand R J, Tsung A, et al. Emerging paradigm: toll-like receptor 4-sentinel for the detection of tissue damage. SHOCK 2006; 26(5): 430-7.
76. Johnson J D, Campisi J, Sharkey C M, et al. Catecholamines mediate stress-induced increases in peripheral and central inflammatory cytokines. NEUROSCIENCE 2005; 135(4): 1295-307.
77. Wang W, Xu M, Zhang Y Y, et al. Fenoterol, a beta(2)-adrenoceptor agonist, inhibits LPS-induced membrane-bound CD14, TLR4/CD14 complex, and inflammatory cytokines production through beta-arrestin-2 in THP-1 cell line. ACTA PHARMACOL SIN 2009; 30(11):1522-8.
78. Du Q, Min S, Chen L Y, et al. Major stress hormones suppress the response of macrophages through down-regulation of TLR2 and TLR4. J SURG RES 2012; 173(2):354-61.
79. Arraes S M, Freitas M S, da Silva S V, et al. Impaired neutrophil chemotaxis in sepsis associates with GRK expression and inhibition of actin assembly and tyrosine phosphorylation. BLOOD 2006; 108(9): 2906-13.
80. Lin W J, Yeh W C. Implication of Toll-like receptor and tumor necrosis factor alpha signaling in septic shock. SHOCK 2005; 24:206-209.
81. Annane D, Trabold F, Sharshar T, et al. Inappropriate sympathetic activation at onset of septic shock: a spectral analysis approach. AM J RESPIR CRIT CARE MED 1999; 160:458-465.
82. Bergquist J, Tarkowski A, Ekman R, et al. Discovery of endogenous catecholamines in lymphocytes and evidence for catecholamine regulation of lymphocyte function via an autocrine loop. PROC NATL ACAD SCI USA 1994; 91(26): 12912-6.
83. Flierl M A, Rittirsch D, Nadeau B A, et al. Phagocyte-derived catecholamines enhance acute inflammatory injury. NATURE 2007; 449(7163):721-5.
84. Mangano D T, Layug E L, Wallace A, et al. Effect of atenolol on mortality and cardiovascular morbidity after noncardiac surgery. Multicenter Study of Perioperative Ischemia Research Group N ENG J MED 1996; 335:1713-1720.
85. Pasternack P F, Grossi E A, Baumann F G, et al. Beta blockade to decrease silent myocardial ischemia during peripheral vascular surgery AM J SURG 1989; 158:113-116
86. Auerbach A D, Goldman L. Beta blockers and reduction of cardiac events in noncardiac surgery. JAMA 2002; 287: 1435-1444.
87. Lidenauer P K, Pekow P, Kaijun W, et al. Perioperative beta blocker therapy and mortality after major noncardiac surgery. N ENG J MED 2005; 353:349-361.
88. Jeschke M G, Norbury W B, et al. Propranolol does not increase inflammation, sepsis, or infectious episodes in severely burned children. J TRAUMA 2007; 62(3):676-81.
89. Lev-Tov H, Dahle S, Moss J et al. Topical Beta-Blocker to Heal Chronic Leg Ulcer. J A M ACAD DERMATOL 2013 (in press).
90. Lembo A. et al., Differential contribution of Toll-like receptors 4 and 2 to the cytokine response to Salmonella enterica serovar Typhimurium and Staphylococcus aureus in mice. INFECT IMMUNITY 2003; 71(10): 6058-62.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. An extracellular matrix scaffold comprising mesenchymal stem cells (MSCs) which have been contacted and/or pre-conditioned with a beta adrenergic receptor antagonist.

2. The extracellular matrix scaffold of claim 1, wherein the MSCs have been cultured in medium comprising a beta adrenergic receptor antagonist.

3. The extracellular matrix scaffold of claim 2, wherein the MSCs have been cultured at least 24 hours in medium comprising a beta adrenergic receptor antagonist.

4. The extracellular matrix scaffold of any one of claims 1 to 3, wherein the MSCs have been cultured under hypoxic conditions.

5. The extracellular matrix scaffold of any one of claims 1 to 4, wherein the antagonist has a Kd for a beta-3 adrenergic receptor that is about 100 or more times greater than a Kd of the antagonist for a non-beta-3 adrenergic receptor.

6. The extracellular matrix scaffold of any one of claims 1 to 4, wherein the beta adrenergic receptor antagonist is a non-selective antagonist for β1 and β2 adrenergic receptors.

7. The extracellular matrix scaffold of claim 6, wherein the beta adrenergic receptor antagonist is selected from carteolol, carvedilol, labetalol, nadolol, penbutolol, pindolol, propranolol, sotalol, timolol, and mixtures, analogs and salts thereof.

8. The extracellular matrix scaffold of any one of claims 1 to 4, wherein the beta adrenergic receptor antagonist is a selective antagonist for β1 adrenergic receptors.

9. The extracellular matrix scaffold of claim 8, wherein the beta adrenergic receptor antagonist is selected from acebutolol, atenolol, betaxolol, bisoprolol, celiprolol, esmolol, metoprolol, nebivolol, and mixtures, analogs and salts thereof.

10. The extracellular matrix scaffold of any one of claims 1 to 4, wherein the beta adrenergic receptor antagonist is a selective antagonist for β2 adrenergic receptors.

11. The extracellular matrix scaffold of claim 10, wherein the beta adrenergic receptor antagonist is selected from butoxamine and ICI-118,551.

12. The extracellular matrix scaffold of any one of claims 1 to 4, wherein the beta adrenergic receptor antagonist is selected from the group consisting of timolol, labetalol, dilevelol, propanolol, carvedilol, nadolol, carteolol, penbutolol, sotalol, ICI-118,551, butoxamine, and mixtures, analogs and salts thereof.

13. The extracellular matrix scaffold of any one of claims 1 to 4, wherein the beta adrenergic receptor antagonist is substantially free of activity as a beta-3 adrenergic receptor agonist.

14. The extracellular matrix scaffold of any one of claims 1 to 13, wherein the beta adrenergic receptor antagonist is cross-linked to the scaffold.

15. The extracellular matrix scaffold of any one of claims 1 to 14, wherein the MSCs are adipose-derived MSCs (Ad-MSCs).

16. The extracellular matrix scaffold of any one of claims 1 to 13, wherein the MSCs are bone-marrow-derived MSCs (BM-MSCs).

17. A kit comprising an extracellular matrix scaffold of any one of claims 1 to 16.

18. A method of promoting, facilitating, and/or increasing healing, closure, re-epithelization and/or dermal regeneration of an epithelial and/or cutaneous wound in a subject in need thereof, comprising placing, implanting, suturing or embedding onto or into the wound an extracellular matrix scaffold of any one of claims 1 to 16.

19. The method of claim 18, wherein the subject has diabetes.

20. The method of any one of claims 18 to 19, wherein the subject is a human.

21. The method of any one of claims 18 to 20, wherein the wound comprises an incision, laceration, abrasion, or ulcer.

22. The method of any one of claims 18 to 21, wherein the wound is a chronic wound.

23. The method of any one of claims 18 to 22, the wound comprises a venous stasis ulcer, a diabetic foot ulcer, a neuropathic ulcer, or a decubitus ulcer.

24. The method of any one of claims 18 to 20, wherein the wound comprises a wound resulting from surgical wound dehiscence.

25. The method of any one of claims 18 to 20, wherein the wound comprises a burn.

26. The method of any one of claims 18 to 24, wherein the epithelial wound comprises skin.

27. The method of any one of claims 18 to 26, wherein the MSCs are autologous to the subject.

28. The method of any one of claims 18 to 26, wherein the MSCs are syngeneic to the subject.

29. The method of any one of claims 18 to 26, wherein the MSCs are allogeneic to the subject.

30. The method of any one of claims 18 to 26, wherein the MSCs are xenogeneic to the subject.

31. The method of any one of claims 18 to 30, wherein the wound is sterile.

32. The method of any one of claims 18 to 30, wherein the wound is not sterile.

33. The method of any one of claims 18 to 32, wherein the beta adrenergic receptor antagonist is applied multiple times to the extracellular matrix scaffold that has been sutured, embedded or implanted into the wound.
